(19) United States
(12) Patent Application Publication
(10) Pub. No.: US 2020/0093815 A1
(43) Pub. Date: Mar. 26, 2020

(54) COMBINATIONS OF EZH2 INHIBITORS WITH FURTHER AGENTS FOR USE IN THE TREATMENT OF CANCER

(71) Applicant: Constellation Pharmaceuticals, Inc., Cambridge, MA (US)

(72) Inventors: Shilpi Arora, Bedford, MA (US); Srividya Balasubramanian, Lexington, MA (US); William D Bradley, Winchester, MA (US); Patrick Trojer, Reading, MA (US)

(21) Appl. No.: 15/747,660

(22) PCT Filed: Jul. 27, 2016
(86) PCT No.: PCT/US2016/044157

§ 371 Date: Jan. 25, 2018

Related U.S. Application Data
(60) Provisional application No. 62/197,863, filed on Jul. 28, 2015.

(51) Int. Cl. A61K 31/4545; A61K 31/4035; A61K 31/573; A61K 31/52; A61K 31/5377; A61K 31/404; A61K 31/496; A61K 31/66; A61K 31/197; A61K 31/453

(52) U.S. Cl. CPC A61K 31/4545; A61K 31/4035; A61K 31/573; A61K 31/52; A61K 31/453; A61K 31/404; A61K 31/496; A61K 31/66; A61K 31/197; A61K 31/5377

(57) ABSTRACT

Provided herein are methods of treating cancer using an effective amount of an EZH2 inhibitor and an effective amount of a second agent. Also provided are compositions comprising an EZH2 inhibitor and an effective amount of a second agent.

### RELATED APPLICATIONS

This application claims priority to U.S. provisional application No. 62/197,863, filed Jul. 28, 2015, the contents of which are incorporated herein in their entirety.

### BACKGROUND

EZH2 (Enhancer of Zeste Homolog 2) is a histone lysine methyltransferase implicated in the pathogenesis of both hematologic and non-hematologic malignancies. EZH2 catalyzes the transfer of one, two and three methyl-groups to lysine 27 of histone 3 (H3K27). EZH2 is the catalytic component of a large, multi-protein complex called polycomb repressive complex 2 (PRC2), which generally functions in transcriptional repression (The Polycomb complex PRC2 and its mark in life. Nature 469, 343-349, 2011). Although in many instances transcriptional silencing by PRC2 is dependent on the catalytic activity of EZH2, it is clear that the physical association of the PRC2 complex with certain genes is also important in transcriptional suppression. The PRC2 complex can alternatively contain a closely related homolog of EZH2, known as EZH1. These two catalytic subunits of the PRC2 complex are the only enzymes known to catalyze H3K27 methylation. In addition to their catalytic activity, EZH1 and EZH2 are multi-domain proteins that mediate other biologic effects through protein-protein and protein-nucleic acid interactions. H3K27 di-methylation and tri-methylation (H3K27me2 and H3K27me3) correlate well with transcriptionally repressed genes, but H3K27 mono-methylation (H3K27me1) is found on transcriptionally active genes (Barski A, et al. (2007) High-resolution profiling of histone methylations in the human genome. Cell;129: 823-837; Ferrari K J, et al. (2014) Polycomb-dependent H3K27me1 and H3K27me2 regulate active transcription and enhancer fidelity. Mol Cell;53:49-62.). Recent genetic studies suggest that EZH1-containing PRC2 controls H3K27me1 levels (Hidalgo I, et al. (2012) Ezh1 is required for hematopoietic stem cell maintenance and prevents senescence-like cell cycle arrest. Cell Stem Cell;11:649-662; Xie H, et al. (2014) Polycomb repressive complex 2 regulates normal hematopoietic stem cell function in a developmental-stage-specific manner. Cell Stem Cell;14:68-80). This is consistent with a putative role of EZH1 in transcriptional elongation (Mousavi K, Zare H, Wang A H, et al. (2012) Polycomb protein Ezh1 promotes RNA polymerase II elongation. Mol Cell;45:255-262). Thus, PRC2-dependent H3K27 methyltransferase activity is implicated in both transcriptional repression and activation, depending on the composition of the complex.

EZH2 (but not EZH1) is frequently overexpressed in human cancer. High levels of expression correlate with increased levels of H3K27me3, late stage disease and poor outcome, for instance in breast, lung, gastric, bladder, ovarian and prostate cancer, leukemia, lymphoma and multiple myeloma (Kleer C G, et al. (2003). EZH2 is a marker of aggressive breast cancer and promotes neoplastic transformation of breast epithelial cells. Proc Natl Acad Sci USA;100:11606-11611; Varambally S, et al. (2002). The polycomb group protein EZH2 is involved in progression of prostate cancer. Nature;419:624-629; Weikert S, et al. (2005) Expression levels of the EZH2 polycomb transcriptional repressor correlate with aggressiveness and invasive potential of bladder carcinomas. Int J Mol Med;16:349-353). A continuously increasing number of functional studies implicate PRC2 and specifically EZH2 in tumorigenesis, cancer progression, metastasis and angiogenesis (Lu C, et al. (2010) Regulation of tumor angiogenesis by EZH2. Cancer Cell;18:185-197; Min J, et al. (2010) An oncogene-tumor suppressor cascade drives metastatic prostate cancer by coordinately activating Ras and nuclear factor-kappaB. Nat Med;16:286-294; Shi J, et al. (2013) The Polycomb complex PRC2 supports aberrant self-renewal in a mouse model of MLL-AF9;Nras(G12D) acute myeloid leukemia. Oncogene;32:930-938; Wilson BG, et al. (2010) Epigenetic antagonism between polycomb and SWI/SNF complexes during oncogenic transformation. Cancer Cell;18:316-328). Recent genomic sequencing studies helped to elucidate the role of EZH2 in germinal center-derived lymphomas. As B-cells exit the germinal center, their EZH2 levels decrease, promoting the expression of genes that ensure terminal differentiation. And conditional expression of an EZH2 mutant allele promotes lymphoid hyperplasia and lymphomagenesis by aberrantly repressing B-cell differentiation genes. The role of EZH2 in the development of germinal center-derived lymphomas has been further substantiated by the discovery of recurrent, monoallelic mutations in the gene encoding EZH2 in 15-25% of germinal center B-cell-like diffuse large B-cell lymphomas (GCB-DLBCL) and in 12-22% of follicular lymphomas (FL) (Ryan R J, et al. (2011) EZH2 codon 641 mutations are common in BCL2-rearranged germinal center B cell lymphomas. PLoS One;6:e28585; Guo S, et al. (2014) EZH2 Mutations in Follicular Lymphoma from Different Ethnic Groups and Associated Gene Expression Alterations. Clin Cancer Res;20:3078-3086; Lohr J G, et al. (2012) Discovery and prioritization of somatic mutations in diffuse large B-cell lymphoma (DLBCL) by whole-exome sequencing. Proc. Natl Acad Sci USA;109:3879-3884; Bodor C, et al. (2011) EZH2 Y641 mutations in follicular lymphoma. Leukemia;25:726-729; Morin R D, et al. (2011) Frequent mutation of histone-modifying genes in non-Hodgkin lymphoma. Nature;476:298-303; Morin R D, et al. (2010) Somatic mutations altering EZH2 (Tyr641) in follicular and diffuse large B-cell lymphomas of germinal-center origin. Nat Genet;42:181-185). Recurrent EZH2 mutations have also been found with low frequency in melanoma (Hodis E, et al. (2012) A landscape of driver mutations in melanoma. Cell;150:251-263). Recurrent mutations in EZH2 affect the amino acid residues Y641, A677 and A687 and alter the substrate specificity of the enzyme, making it more efficient in the conversion of H3K27 from a di-methylated to a tri-methylated state (Majer C R, et al. (2012) A687V EZH2 is a gain-of-function mutation found in lymphoma patients. FEBS Lett;586(19):3448-51; McCabe M T, et al. (2012a) Mutation of A677 in histone methyltransferase EZH2 in human B-cell lymphoma promotes hypertrimethylation of histone H3 on lysine 27 (H3K27). Proc Natl Acad Sci USA;109:2989-2994; Sneeringer C J, et al. (2010) Coordinated activities of wild-type plus mutant EZH2 drive tumor-associated hypertrimethylation of lysine 27 on histone H3 (H3K27) in human B-cell lymphomas. Proc Natl Acad Sci USA;107:20980-20985; Wigle T J, et al. (2011) The Y641C mutation of EZH2 alters substrate specificity for histone H3 lysine 27 methylation states. FEBS Lett 585, 3011-3014; Yap D B, et al. (2011) Somatic mutations at EZH2 Y641 act dominantly through a mechanism of selectively altered PRC2 catalytic activity, to increase H3K27 trimethylation. Blood;117: 2451-2459). Consequently, malignant cells containing one of these mutations exhibit higher global levels of H3K27me3 than those with the wild-type enzyme. The dependence of these lymphomas on the heightened catalytic activity of the mutated enzyme is reflected in their sensitivity to highly selective inhibitors of EZH2 (Bradley, W. D. et al. EZH2 Inhibitor Efficacy in Non-Hodgkin's Lymphoma Does Not Require Suppression of H3K27 Monomethylation. Chem. Biol. 21, 1463-1475 (2014); Garapaty-Rao S, et al. (2013) Identification of EZH2 and EZH1 small molecule inhibitors with selective impact on diffuse large B cell lymphoma cell growth. Chem Bio1;20:1329-1339; Knutson SK, et al. (2012) A selective inhibitor of EZH2 blocks H3K27 methylation and kills mutant lymphoma cells. Nat Chem Bio1;8:890-896; McCabe M T, et al. (2012b) EZH2 inhibition as a therapeutic strategy for lymphoma with EZH2-activating mutations. Nature;492:108-112; Qi W, et al. (2012) Selective inhibition of Ezh2 by a small molecule inhibitor blocks tumor cells proliferation. Proc Natl Acad Sci USA;109: 21360-21365; Konze K D, et al. (2013) An Orally Bioavailable Chemical Probe of the Lysine Methyltransferases EZH2 and EZH1. ACS Chem Bio1;8(6):1324-1334; Nasveschuk C G, et al. (2014) Discovery and Optimization of Tetramethylpiperidinyl Benzamides as Inhibitors of EZH2. ACS Med Chem Lett;5:378-383; Knutson S K, et al. (2014) Selective Inhibition of EZH2 by EPZ-6438 Leads to Potent Antitumor Activity in EZH2 Mutant Non-Hodgkin Lymphoma. Mol Cancer Ther;13:842-854). While the anti-tumor activity of EZH2 inhibitors is most consistently observed in models of lymphoma with activating mutations in EZH2, there are models of lymphoma and other malignancies that are sensitive to EZH2 inhibition but that contain only wild-type EZH2 (Bradley, W. D. et al. EZH2 Inhibitor Efficacy in Non-Hodgkin's Lymphoma Does Not Require Suppression of H3K27 Monomethylation. Chem. Biol. 21,1463-1475 (2014); McCabe M T, et al. (2012b) EZH2 inhibition as a therapeutic strategy for lymphoma with EZH2-activating mutations. Nature;492:108-112).

EZH2 is regarded as an oncogene in certain cancer types. Efficacy in models of hematological malignancies and solid tumors has been shown with pharmacological inhibition of EZH2. See e.g., WO 2013/120104 and WO 2014/124418. Given its role in the regulation of diverse biological processes, and the therapeutic benefits associated with its inhibition, EZH2 remains an attractive target for modulation.

### SUMMARY

It has now been found that administration of an EZH2 inhibitor and other therapeutic agents synergistically treat cancer. See e.g., FIG. 1 and FIG. 2, which illustrates tumor regression upon administration of a combination of an EZH2 inhibitor and CHOP; FIG. 3 and FIG. 4, which illustrates synergy in OPM-2 and KMS27 multiple myeloma cells with an EZH2 inhibitor and lenalidomide; FIGS. 7-9, which illustrates synergy in lymphoma cells with an EZH2 inhibitor and idelalisib and buparlisib; FIG. 10 and FIG. 11, which illustrates synergy between EZH2 inhibitor and an HDAC inhibitor (Panobinostat) in multiple myeloma cells in vitro and in vivo; FIG. 12, which illustrates sensitivity of leukemia cells to the combination of an EZH2 inhibitor and an LSD1 inhibitor; FIG. 13, which shows synergy between EZH2 inhibitor and dexamethasone in a multiple myeloma in vivo model; FIG. 14, which illustrates synergy between EZH2 inhibitor and Melphalan in multiple myeloma cells; FIG. 15, which illustrates the synergy between EZH2 inhibitor and Bortezomib in multiple myeloma cells; FIG. 16 which shows combinatorial activity of EZH2 inhibitor with standard of care agents (Lenalidomide, Bortezomib, Panobinostat and Melphalan) in multiple myeloma cells; and FIG. 17 and FIG. 18 which shows combinatorial activity of EZH2 inhibitor and Ibrutinib in lymphoma cell lines.

Based on these results, provided herein are methods of treating a subject with cancer by administering to the subject an effective amount of an EZH2 inhibitor and an effective amount of a second agent selected from phthalimides; anti-inflammatory steroids; PI3K inhibitors; LSD1 inhibitors; histone deacetylase inhibitors; cyclophosphamide, hydroxydaunorubicin, oncovin, and prednisone (CHOP); rituximab and CHOP; prednisolone; bortezomib; bromodomain-targeted compounds; and ibrutinib.

Based on these results, also provided herein are methods of treating a subject with cancer by administering to the subject an effective amount of an EZH2 inhibitor and an effective amount of an alkylating antineoplastic agent.

Also provided herein are pharmaceutical compositions comprising an EZH2 inhibitor and an additional therapeutic agent described herein.

### BRIEF DESCRIPTION OF THE FIGURES

FIG. 1 illustrates the synergistic effect of an EZH2 inhibitor together with CHOP.

FIG. 2 illustrates the synergistic effect of an EZH2 inhibitor together with CHOP.

FIG. 3 illustrates the synergistic effect of an EZH2 inhibitor with lenalidomide in OPM-2 multiple myeloma cells, where FIG. 3A illustrates the average percentage of cells in drug-treated wells as compared to DMSO-treated cells from independent duplicate experiments (i and ii), FIG. 3B illustrates isobologram plots for the combination, FIG. 3C and FIG. 3D illustrate bliss independence and Loewe excess score, FIG. 3E illustrates a dose-dependent effect of an EZH2 inhibitor alone, and FIG. 3F illustrates the dose response curves to show synergy between EZH2 inhibitor and lenalidomide.

FIG. 4 illustrates the synergistic effect of an EZH2 inhibitor together with lenalidomide in KMS-27 multiple myeloma cells, where FIG. 4A illustrates the average percentage of cells in drug-treated wells as compared to DMSO-treated cells from independent duplicate experiments (i and ii), FIG. 4B illustrates isobologram plots for the combination, FIG. 4C and FIG. D illustrate bliss independence and Loewe excess score, FIG. 4E illustrates a dose-dependent effect of an EZH2 inhibitor alone, and FIG. 4F illustrates the dose response curves to show synergy between EZH2 inhibitor and lenalidomide. FIG. 5 illustrates the summary of the synergistic effect of an EZH2 inhibitor together with lenalidomide across a panel of multiple myeloma cells.

FIG. 6 illustrates the synergistic reduction in tumor burden in an in vivo xenograft model of OPM-2 cells, wherein mice were treated with a combination of EZH2 inhibitor and lenalidomide, where FIG. 6A illustrates an in vivo combination experiment using an OPM-2 subcutaneous xenograft BALB/C nude mouse model, FIG. 6B illustrates body weight change, and FIG. 6C illustrates global H3K27me3 and total H3 levels in the tumor samples at the end of the study.

FIG. 7 illustrates the synergistic effect of an EZH2 inhibitor with idelalisib (CAL-101) in KARPAS-422 lymphoma cells.

FIG. 8 illustrates the synergistic effect of an EZH2 inhibitor with buparlisib (BKM-120) in the KARPAS-422 lymphoma cells.

FIG. 9 illustrates the synergistic effect of an EZH2 inhibitor together idelalisib (CAL-101), where FIG. 9A is in Pfeiffer lymphoma cells, FIG. 9B is in SU-DHL-6 lymphoma cells, and FIG. 9C is in WSU-DLCL2 lymphoma cells.

FIG. 10A illustrates the synergistic effect of an EZH2 inhibitor with HDAC inhibitor (Panobinostat) in 19 multiple myeloma cells. FIG. 10B and FIG. 10C illustrate the synergistic effect of an EZH2 inhibitor together with Panobinostat in vitro.

FIG. 11 illustrates the synergy between EZH2 inhibitor and Panobinostat in vivo in an OPM-2 xenograft model, where FIG. 11A shows the tumor size in the variaous arms of the study, FIG. 11B shows the assessment of body weight changes in the mice and FIG. 11C shows the assessment of H3K27me3 and total H3 levels in tumors at the end of the study.

FIG. 12 illustrates the synergistic effect of an EZH2 inhibitor with an LSD1 inhibitor in leukemia cells, where FIG. 12A illustrates the summary of AML cell line viability data, FIG. 12B shows the data analysis of the EZH2 inhibitor and LSD1 inhibitor combination in MOLM-13 cells and FIG. 12C shows the synergy between these inhibitors in other leukemia cell lines.

FIG. 13 illustrates the synergistic reduction in tumor burden in an in vivo xenograft model of OPM-2 cells wherein mice were treated with a combination of EZH2 inhibitor and dexamethasone, where FIG. 13A illustrates reduction in tumor size and FIG. 13B illustrates % change in bodyweight.

FIG. 14 illustrates the assessment of synergistic effect of EZH2 inhibitor with Melphalan in multiple myeloma cell lines, where FIG. 14A shows the summary of all multiple myeloma cell viability data, FIG. 14B and FIG. 14C show the synergistic dose response curves in KMS-34 and MM-1R multiple myeloma cell lines, respectively.

FIG. 15 illustrates the assessment of synergy between EZH2 inhibitor and Bortezomib in multiple myeloma cell lines, where FIG. 15A shows the summary of all multiple myeloma cell line viability data, FIG. 15B and FIG. 15C show the synergistic dose response curve in U266 multiple myeloma cell line.

FIG. 16 summarizes the asessment of combinatorial activity between EZH2 inhibitor and standard of care agents in multiple myeloma.

FIG. 17 summarizes the synergy between EZH2 inhibitor and ibrutinib in lymphoma cell lines.

FIG. 18 illustrates the synergy between EZH2 inhibitor and ibrutinib in KARPAS-422 and SUDHL-5 cell lines.

### DETAILED DESCRIPTION

In one aspect, the present disclosure provides a method of treating cancer in a subject in need thereof, comprising the step of administering to the subject in need thereof an effective amount of an EZH2 inhibitor and an effective amount of a second agent selected from a phthalimide; an anti-inflammatory steroid; a PI3K inhibitor; an LSD1 inhibitor; a histone deacetylase inhibitor; cyclophosphamide, hydroxydaunorubicin, oncovin, and prednisone (CHOP); rituximab and CHOP; prednisolone; bortezomib; a bromodomain-targeted compound; and ibrutinib. Alternatively, the methods described herein comprise an effective amount of an EZH2 inhibitor together with a second agent selected from a phthalimide, an anti-inflammatory steroid, a PI3K inhibitor, an LSD1 inhibitor, a histone deacetylase (HDAC) inhibitor, and CHOP.

It will be understood that unless otherwise indicated, the administrations described herein include administering a described EZH2 inhibitor prior to, concurrently with, or after administration of a second agent described herein. Thus, simultaneous administration is not necessary for therapeutic purposes. In one aspect, however, the EZH2 inhibitor is administered concurrently with the second agent.

As described herein, a phthalimide means a small molecule that elecits anti-cancer properties and comprises a phthalimide moiety as part of the scaffold. Examples of phthalimides include, but are not limited to, lenalidomide, thalidomide, pomalidomide, and those described in WO 2013/130849, WO 2010/056344, WO2012/015986, and WO2012/079022. In one aspect, the methods described herein comprise an effective amount of an EZH2 inhibitor together with a phthalimide selected from lenalidomide, thalidomide, and pomalidomide. In one alternative aspect, the methods described herein comprise an effective amount of an EZH2 inhibitor together with the phthalimide lenalidomide.

As described herein, an anti-inflammatory steroid means a steroid that has anti-inflammatory properties. Examples include, but are not limited to, amcinonide, betamethosone diproprionate, clobetasol, clocortolone, dexamethasone, diflorasone, dutasteride, flumethasone pivalate, flunisolide, fluocinolone acetonide, fluocinonide, fluorometholone, fluticasone propionate, flurandrenolide, and hydroflumethiazide. In one aspect, the pharmaceutical compositions described herein comprise an effective amount of an EZH2 inhibitor together with the anti-inflammatory steroid dexamethasone.

As described herein, a PI3K inhibitor means a compound that functions by inhibiting one or more of the phosphoinositide 3-kinase enzymes. Examples include, but are not limited to, idelalisib, buparlisib, A-769662, Afuresertib, AMG-319, ARQ-092, AS-252424, AS-604850, AS-605240, AZD6482, BAY 80-6940, BEZ235, BGT-226, BYL719, CAY10505, CC-115, CC-223, CH5132799, Copanlisib, CUDC-907, CZC24832, D-106669, D-116883, D-87503, Deguelin, DS-3078a, Duvelisib, Everolimus, GDC-0032, GDC-0349, GDC-0980, GSK1059615, GSK2126458, GSK-2141795, HS-173, IC-87114, INCB040093, INK1117, LY2780301, LY294002, MK-2206, MLN0128, NU7441, OSI-027, Panulisib, PF-04691502, PF376304, Phenformin hydrochloride, PI-103, Pictilisib, PIK-124, PIK-294, PIK-39, PIK-90, PIK-93, PKI-402, PKI-587, PP121, PWT33597, PX-866, Quercetin, Ridaforolimus, Rigosertib, RP-6530, SAR245408, SAR260301, SF1126, SF1326, Sirolimus, Staurosporine, TASP0415914, Temsirolimus, TG100-115, TGR-1202, TGX221, Theophylline, Triciribine, VS-5584, Wortmannin, XL-765, and ZSTK474. In one aspect, the pharmaceutical compositions described herein comprise an effective amount of an EZH2 inhibitor together with the PI3K inhibitor idelalisib or buparlisib.

As described herein, a histone deacetylase inhibitor means a compound that interferes with the fuction of histone deacetylase. Examples include, but are not limited to, Vorinostat, Romidepsin, Chidamide, Panobinostat, Valproic acid, Belinostat, Mocetinostat, Abexinostat, Entinostat, SB939, Resminostat, Givinostat, Quisinostat, Kevetrin, CUDC-101, AR-42, CHR-2845, CHR-3996, 4SC-202, CG200745, ACY-1215, ME-344, and sulforaphane. In one aspect, the pharmaceutical compositions described herein comprise an effective amount of an EZH2 inhibitor together with the histone deacetylase inhibitor, Panobinostat.

As described herein, an LSD1 inhibitor means a compound that modulates the activity of lysine-specific demethylase. Examples include, but are not limited to, those described in U.S. Provisional Application No. 62/151,706, GSK2879552, ORY-1001, RG6016, U.S. Pat. No.8,853,408, WO 2011/035941, WO 2011/131697, WO 2012/013727, WO 2012/013728, WO 2012/107498, WO 2012/107499, and WO 2012/135113. In one aspect, the methods described herein comprise an effective amount of an EZH2 inhibitor together with the LDS1 inhibitor [RN-1] (1-(4-methyl-l-piperazinyl)-2-[[(1R*,2S*)-2-[4-phenylmethoxy)phenyl]cyclopropyl]amino]ethanone dihydrochloride), ORY-1001 (N1-((1R,2S)-2-phenylcyclopropyl)cyclohexane-1,4-diamine hydrochloride), or GSK2879552 (4-((4-((((1R,2S)-2-phenylcyclopropyl)amino)methyl)piperidin-1-yl)methyl)benzoic acid hydrochloride). In one aspect, the methods described herein comprise an effective amount of an EZH2 inhibitor together with the LDS1 inhibitor [RN-1]. See Neelamegam R, Ricq E L, Malvaez M, Patnaik D, Norton S, Carlin S M, et al. Brain-penetrant LSD1 inhibitors can block memory consolidation. ACS Chem Neurosci. 2012;3:120-8.

As used herein, “alkylating antineoplastic agent” means a compound used in cancer treatment that attaches an alkyl group (CₙH₂ₙ₊₁) to DNA. Examples include, but are not limited to, nitrogen mustards (e.g., cyclophosphamide, mechlorethamine, mustargen, uramustine, melphalan, chlorambucil, ifosfamide, and bendamustine), nitrosoureas (e.g., carmustine, lomustine, and streptozocin), alkyl sulfonates (e.g., busulfan), plantinum-based compounds (e.g., cisplatin, carboplatin, nedaplatin, oxaliplatin, and satraplatin, and others. In one aspect, the methods described herein comprise an effective amount of an EZH2 inhibitor together with the alkylating antineoplastic agent melphalan.

In one aspect, the methods described herein comprise an effective amount of an EZH2 inhibitor together with CHOP.

EZH2 inhibitors in the present methods include e.g., a small molecule or biologic that is capable of inhibiting EZH2 methyltransferase activity. Inhibition can be measured in vitro, in vivo, or from a combination thereof. In one aspect, the EZH2 inhibitors are selected from EPZ005687, EPZ011989, EI1, GSK126, GSK343, UNC1999, and EPZ-6438, as well as from those described in WO 2013/075083, WO 2013/075084, WO 2013/078320, WO 2013/120104, WO 2014/124418, WO 2014/151142, and WO 2015/023915. In one alternative aspect, the EZH2 inhibitors in the the methods described herein are selected from

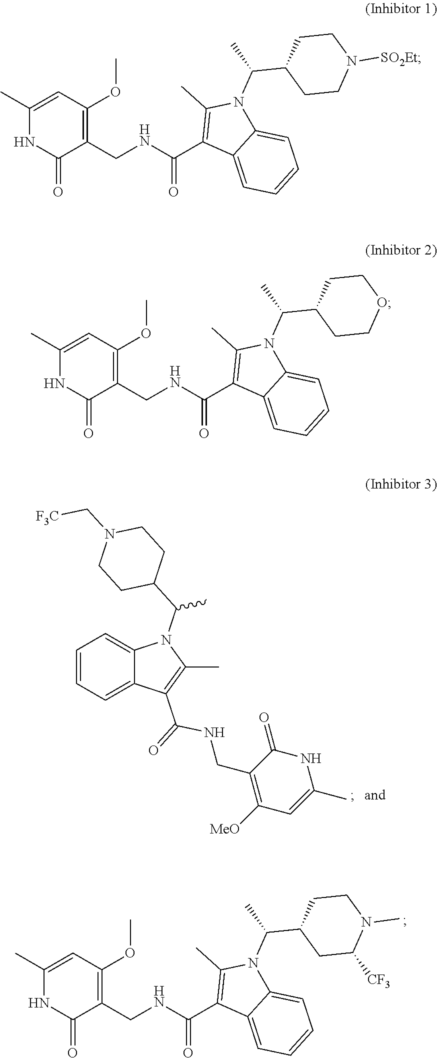

or a pharmaceutically acceptable salt thereof. In another alternative aspect, the EZH2 inhibitors are

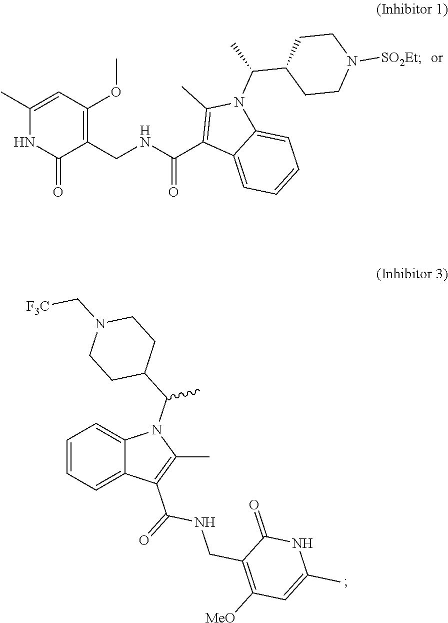

or a pharmaceutically acceptable salt thereof. In another alternative aspect, the EZH2 inhibitors are

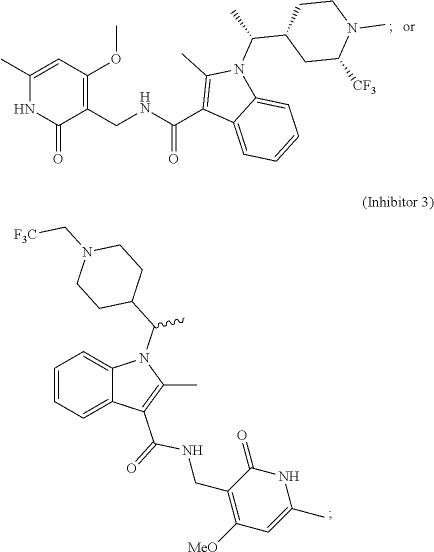

or a pharmaceutically acceptable salt thereof.

As described herein, the amount of an EZH2 inhibitor and a second agent as described herein is such that together, they elicit a synergistic effect to measurably modulate a histone methyl modifying enzyme, inhibit EZH2 and/or treat one or more cancers as described herein in a biological sample or in a patient.

As used herein, the terms “treatment,” “treat,” and “treating” refer to reversing, alleviating, or inhibiting the progression of a cancer, or one or more symptoms thereof, as described herein. Exemplary types of cancer include e.g., adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyo sarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the cancer treated by the combination of an EZH2 inhibitor (e.g., those described in paragraph [0027]) and second agent as described herein is selected from melanoma, prostate cancer, breast cancer, colon cancer, ovarian cancer, bladder cancer, lung adenocarcinoma, and carcinoma of the pancreas. In another aspect, the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL). In another aspect, the cancer treated is selected from Hodgkin's lymphoma, non-Hodgkin's lymphoma, chronic lymphocytic leukemia, and multiple myeloma. In another aspect, the cancer treated is non-Hodgkin's lymphoma.

In a further aspect, the cancer treated by the combination of an EZH2 inhibitor described herein (e.g., those described in paragraph [0027]) and second agent selected from lenalidomide and dexamethasone is multiple myeloma. In an even further aspect the cancer treated by the combination of an EZH2 inhibitor described herein (e.g., those described in paragraph [0026]) and the LSD1 inhibitor [RN-1] in multiple myeloma or adult acute myeloid leukemia (AML). In an even further aspect, the cancer treated by the combination of an EZH2 inhibitor described herein (e.g., those described in paragraph [0026]) and idelalisib, buparlisib, or CHOP is a lymphoma.

Pharmaceutical compositions comprising an EZH2 inhibitor and second agent as described herein are also included.

Also included are the use of an EZH2 inhibitor and a second agent as described herein in the manufacture of a medicament for the treatment of one or more cancers described herein. Also included herein are pharmaceutical compositions comprising an EZH2 inhibitor and a second agent as described herein optionally together with a pharmaceutically acceptable carrier, in the manufacture of a medicament for the treatment of one or more cancers described herein. Also included is an EZH2 inhibitor for use in combination with a second agent as described herein for the treatment of a subject with cancer. Further included are pharmaceutical compositions comprising an EZH2 inhibitor and a second agent as described herein, optionally together with a pharmaceutically acceptable carrier, for use in the treatment of one or more cancers described herein. Futher included are pharmaceutical compositions comprising an EZH2 inhibitor and a second agent as described herein optionally together with a pharmaceutically acceptable carrier for use in the treatment of one or more cancers described herein.

The term “pharmaceutically acceptable carrier, adjuvant, or vehicle” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Other forms of administration are as described in WO 2013/075083, WO 2013/075084, WO 2013/078320, WO 2013/120104, WO 2014/124418, WO 2014/151142, and WO 2015/023915, the contents of which are incorporated herein by reference.

### EXEMPLIFICATION

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

Inhibitor 1 was prepared according to the procedures described in Bradley et al, 2014 above. Inhibitor 3 was prepared according to the procedures described in WO 2013/120104.

### Synergism Between EZH2 Inhibitors and CHOP

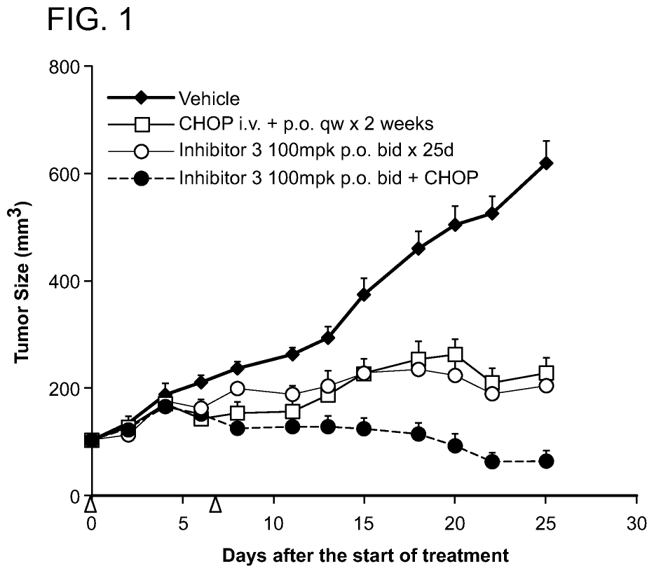

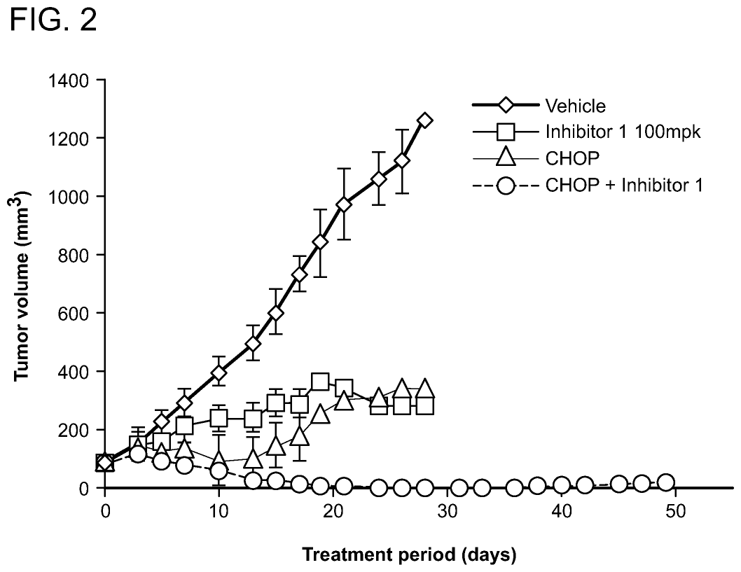

As illustrated in FIG. 1, a combination of Inhibitor 3 and CHOP resulted in tumor regression in a KARPAS-422 xenograft model. KARPAS-422 xenograft mice were treated with vehicle, 100 mg/kg Inhibitor 3, (po, BID), CHOP, or a combination of both regimens. CHOP was dosed twice, at day 0 and day 6 and then ceased due to body weight loss. Inhibitor 3 did not show any adverse effects and did not add to CHOP toxicity. Similarly, as illustrated in FIG. 2, tumor volume measurements were significantly less with a combination of Inhibitor 1 and CHOP. Nude mice with KARPAS-422 xenograft were treated with vehicle, 100 mg/kg Inhibitor 1, sc, BID, CHOP, or a combination of both regimens. Tumor volume measurements were recorded at indicated days post initial dose.* CHOP: Cyclophosphamide, 40 mg/kg, i.v.; doxorubicin, 3.3 mg/kg, i.v.; vincristine, 0.5 mg/kg, i.v.; and prednisone, 0.2 mg/kg, every day for 5 days, p.o.

Synergism Between EZH2 Inhibitors and Phthalimides (Lenalidomide)

For all these experiments estimating the combinatorial response between EZH2 inhibitor and lenalidomide, cells were pre-treated with 8 different doses of EZH2 inhibitor for 8 days (Cells were split after 4 days and plated onto EZH2 inhibitor containing plates for an additional 4 days). After pre-treatment, cells were split and seeded onto EZH2 inhibitor and lenaldomide containing plates (compounds plated in a matrix format). Viability was assessed using Cell Titre Glo after 4 days of co-treatment.

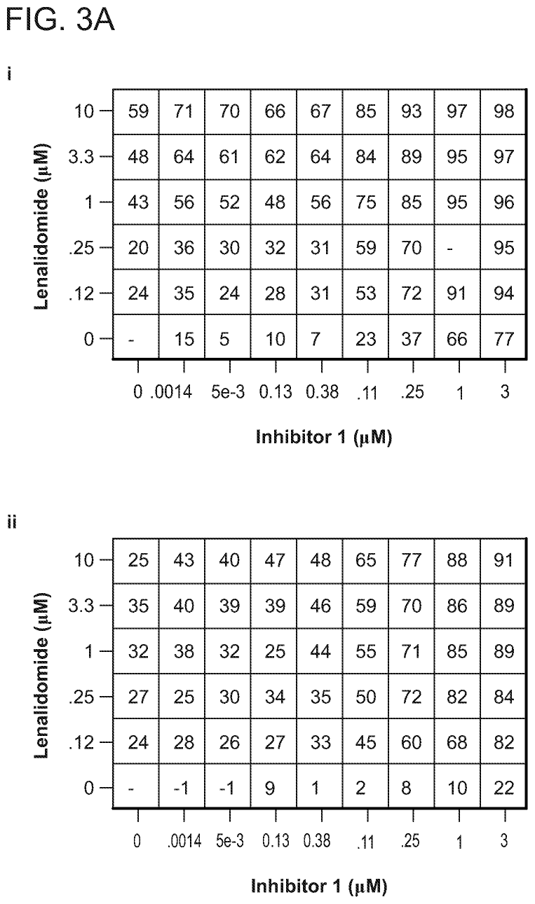

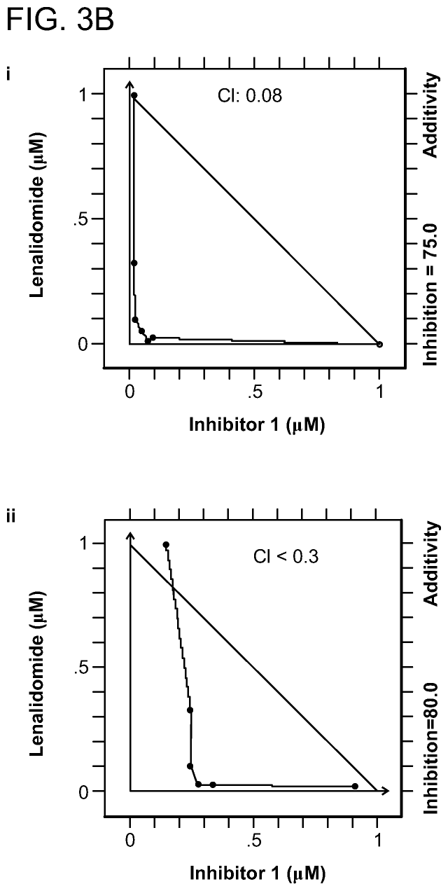

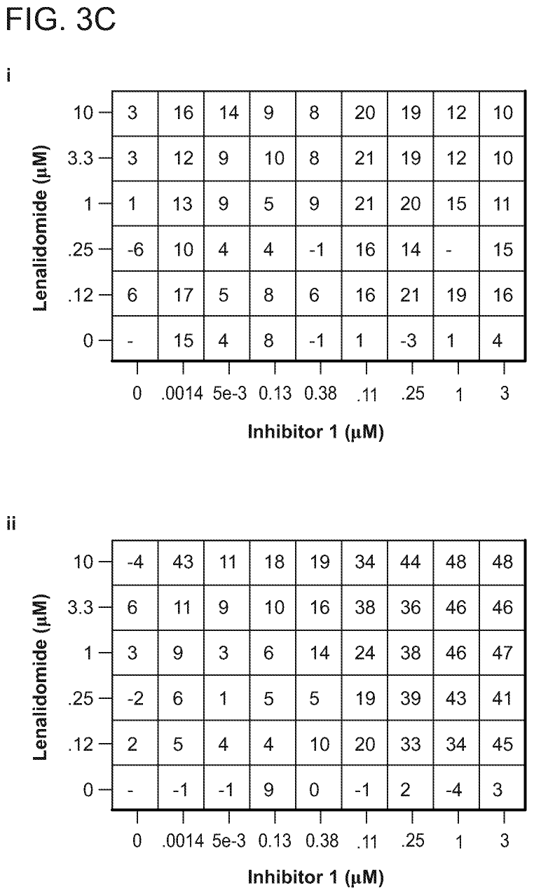

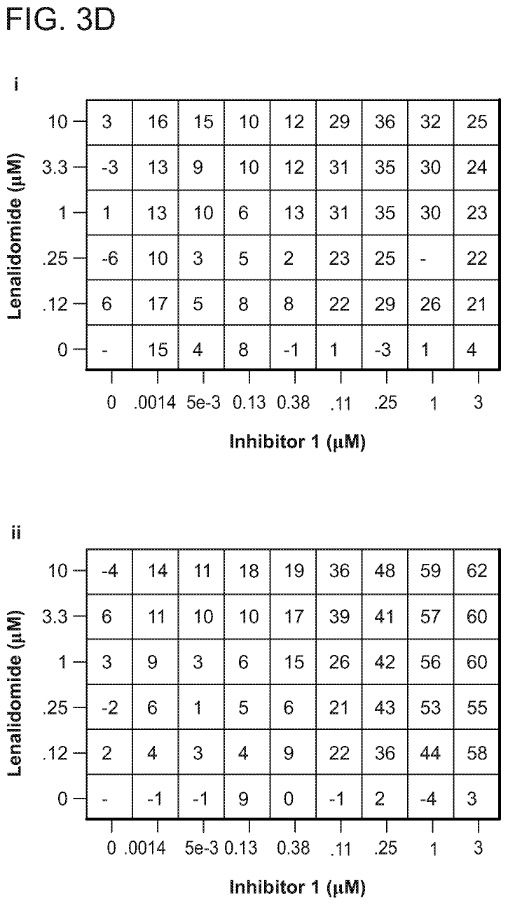

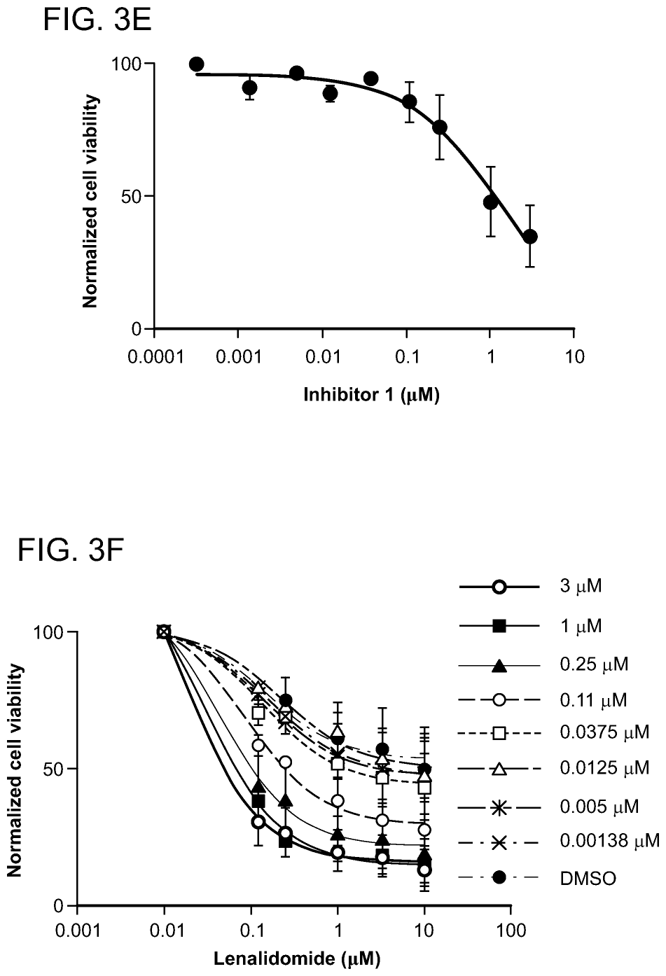

As illustrated by FIG. 3, Inhibitor 1 and lenalidomide shows synergy in OPM-2 multiple myeloma cells. Data are represented as the average percentage of cells in drug-treated wells as compared to DMSO-treated wells from independent duplicate experiments (i and ii). See FIG. 3A. To assess synergy, the data was analyzed in a number of ways including calculation of combination index, bliss independence score and Loewe excess score. Isobologram plots (based on Chou-Talalay method) to illustrate significant synergy in the two independent replicate experiments (i and ii). See FIG. 3B. Bliss independence score for the two independent replicates (i and ii) and Loewe excess score for the two independent replicates (i and ii) were determined and suggest a synergistic activity profile of the combination as compared to single agent activity. See FIG. 3C and FIG. D. The graph shows a dose-dependent effect of Inhibitor 1 alone on cell viability. See FIG. 3E. Data are represented as the mean of independent duplicate experiments ±s.d. See FIG. 3F. The graph shows the effect of lenalidomide as a single agent or in combination with Inhibitor 1. For this plot, the single agent activity of Inhibitor 1 was subtracted to illustrate the synergistic effects of increasing doses of Inhibitor 1 when combined with lenalidomide.

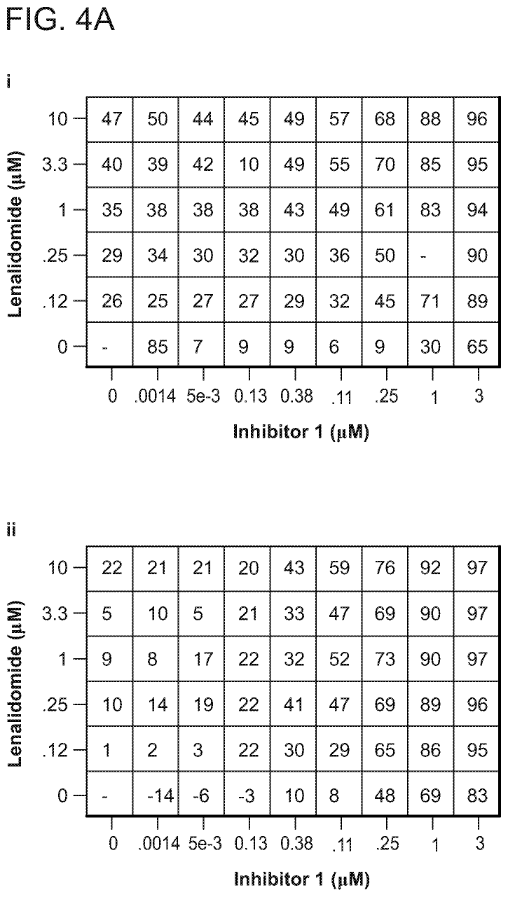

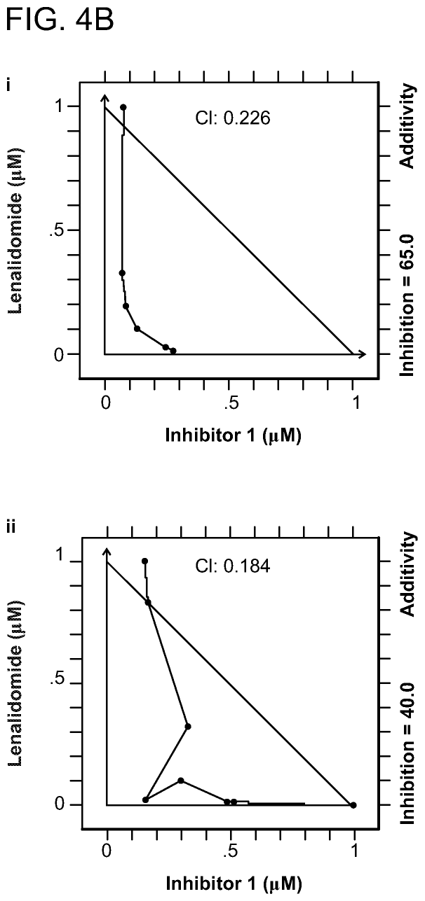

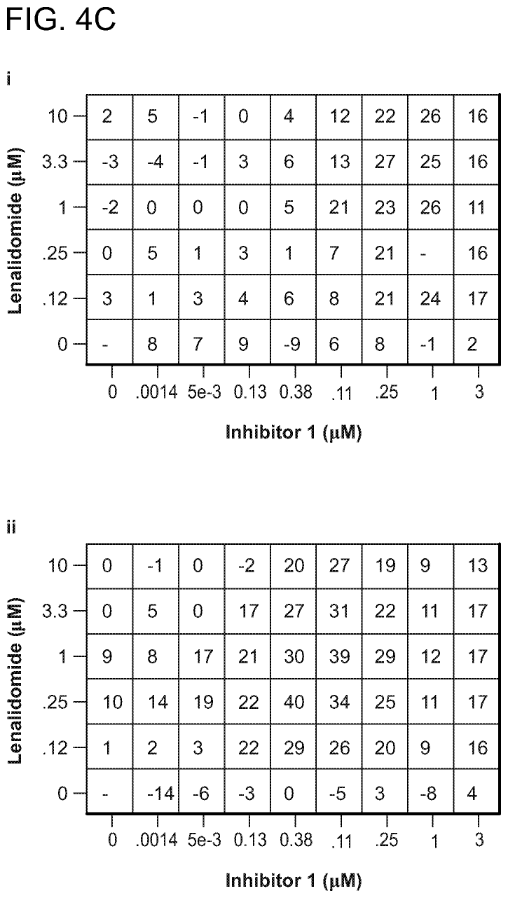

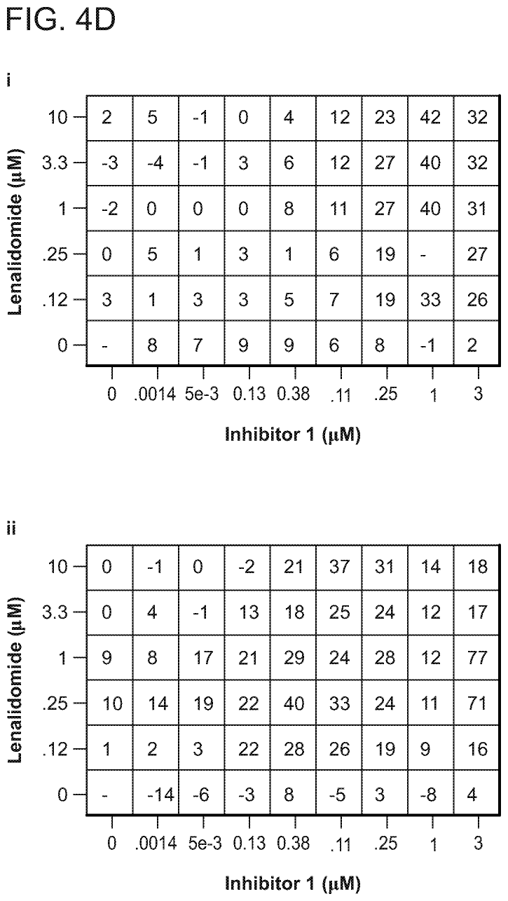

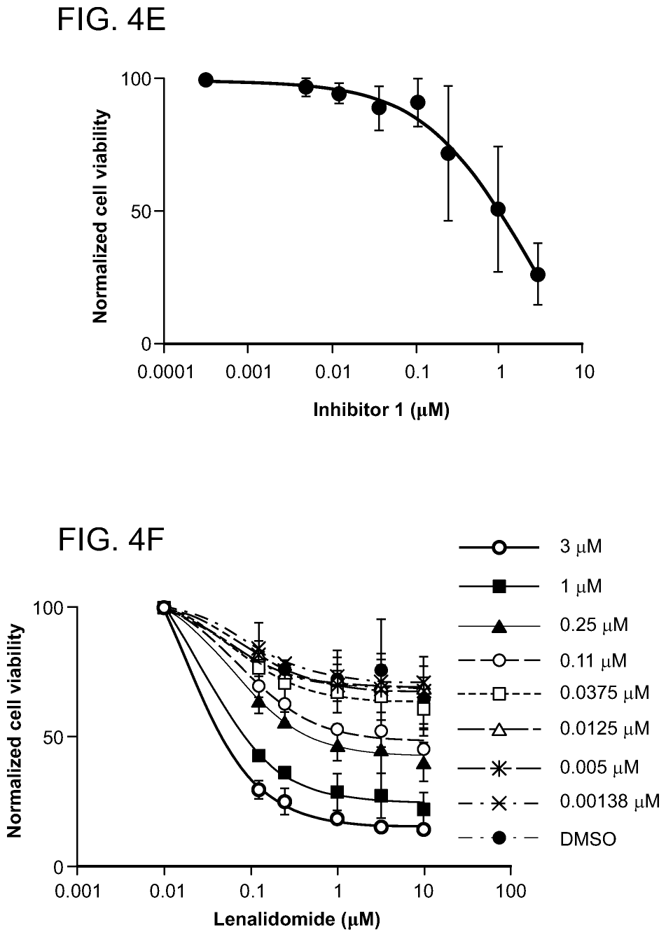

As illustrated by FIG. 4, Inhibitor 1 and lenalidomide shows synergy in KMS-27 multiple myeloma cells. Data are represented as the average percentage of cells in drug-treated wells as compared to DMSO-treated wells from independent duplicate experiments (i and ii). See FIG. 4A. To assess synergy, the data was analyzed in a number of ways including calculation of combination index, bliss independence score and Loewe excess score. Isobologram plots (based on Chou-Talalay method) to illustrate significant synergy in the two independent replicate experiments (i and ii). See FIG. 4B. Bliss independence score for the two independent replicates (i and ii) and Loewe excess score for the two independent replicates (i and ii) were determined and suggest a synergistic activity profile of the combination as compared to single agent activity. See FIG. 4C and FIG. D. The graph shows a dose-dependent effect of Inhibitor 1 alone on cell viability. See FIG. 4E. Data are represented as the mean of independent duplicate experiments ±s.d. See FIG. 4F. The graph shows the effect of lenalidomide as a single agent or in combination with Inhibitor 1. For this plot, the single agent activity of Inhibitor 1 was subtracted to illustrate the synergistic effects of increasing doses of Inhibitor 1 when combined with lenalidomide.

The potential synergy of Inhibitor 1 and lenalidomide was assessed across a panel of 19 multiple myeloma cell lines. FIG. 5 summarizes the response to Inhibitor 1 and lenalidomide used as single agents or in combination. Bliss excess volume (see below) for each cell line was calculated to rank order the cell lines. In the right column, the shift in GI50 values for lenalidomide are shown when 3 μM Inhibitor 1 is added. This shift in GI50 is independent of single agent activity of Inhibitor 1 at this concentration (single agent activity of Inhibitor 1 has been subtracted). Data are represented as the mean of independent duplicate experiments±s.d. The data show that synergy is not limited to cell lines that respond to both inhibitors.

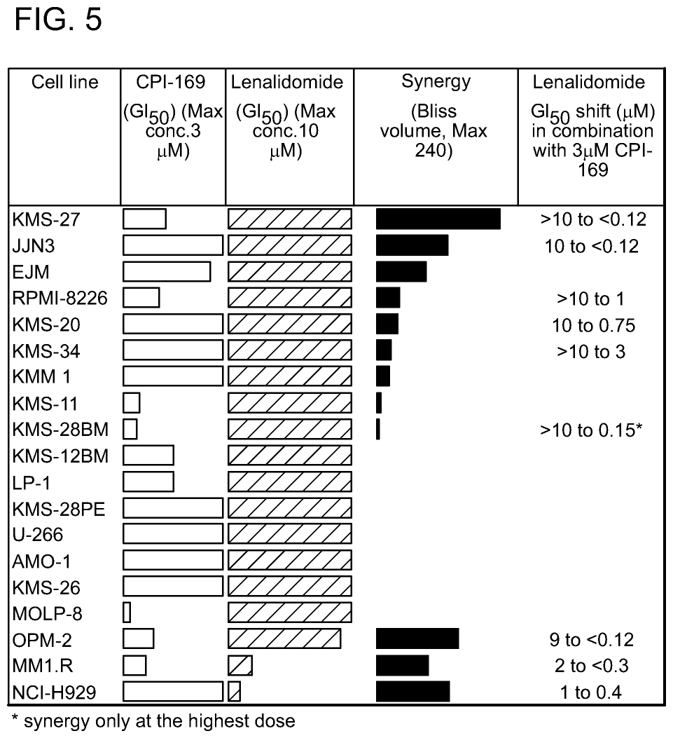

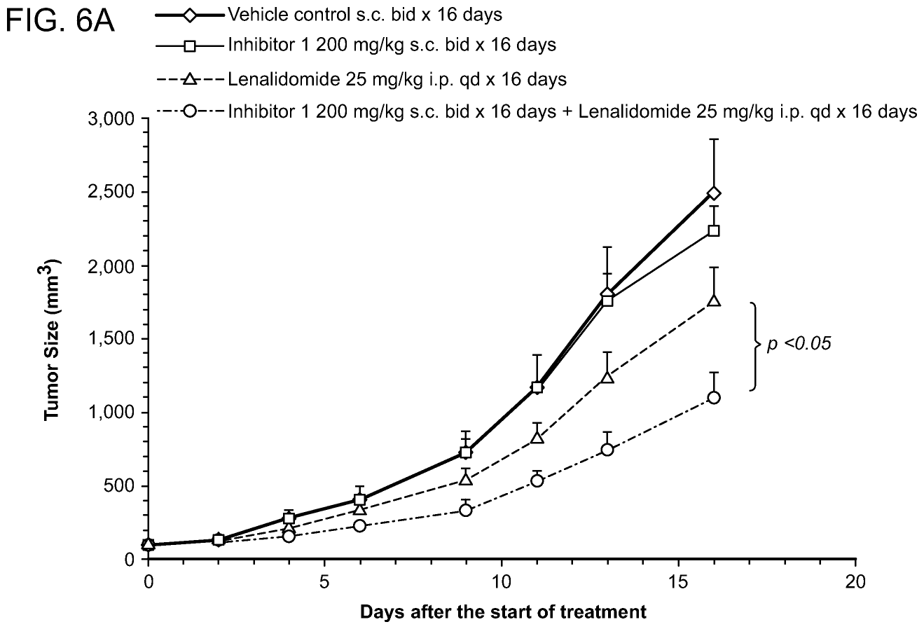

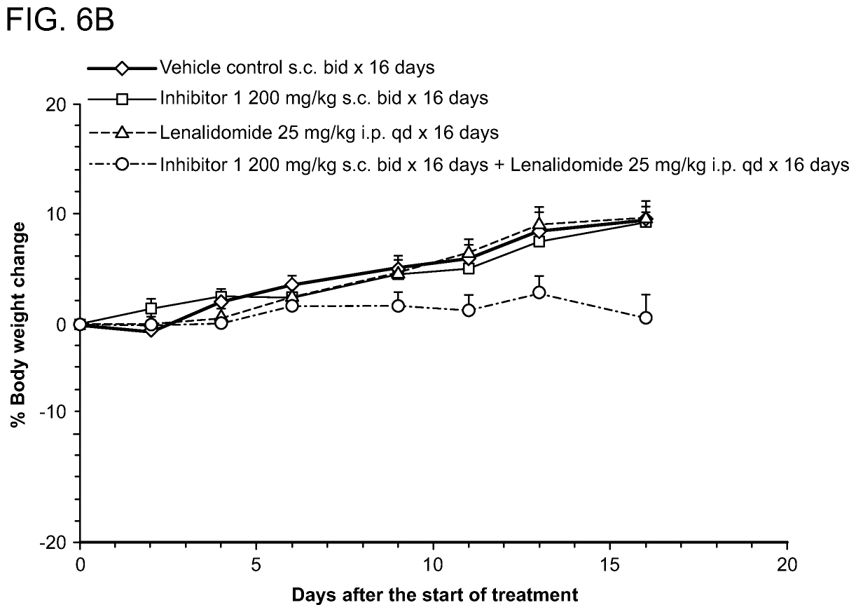

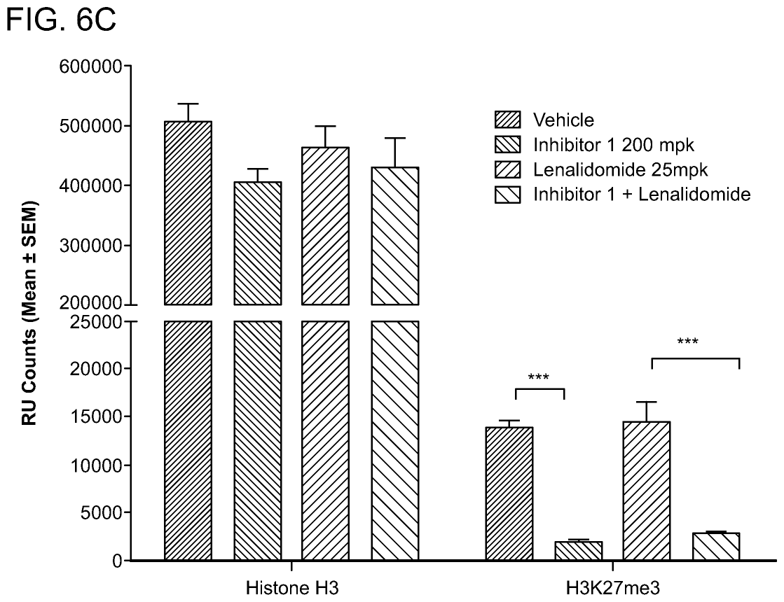

EZH2 Inhibitor 1 and lenalidomide act synergistically and reduce tumor growth in the OPM-2 xenograft model. See FIG. 6 where FIG. 6A is an in vivo combination experiment using an OPM-2 subcutaneous xenograft BALB/C nude mouse model (n=8 per cohort). The experiment was carried out to assess the impact of Inhibitor 1 and lenalidomide in combination. Inhibitor 1 was dosed twice daily at 200 mg/kg, administered subcutaneously (200 mpk SC BID) and lenalidomide was dosed intraperitoneally once a day at 25 mg/kg (25 mpk IP QD) for the course of the entire study. Tumor volume was plotted as a function of time. Data are represented as the mean tumor volume per cohort and time point ±s.d. Bodyweight was assessed for vehicle and compound-treated animals for the experiment described in FIG. 6A. No significant adverse effects were observed during the course of the experiment. See FIG. 6B. Data are represented as described in FIG. 6A. Vehicle and compound-treated tumors were collected at the end of the study and analyzed for global H3K27me3 and total H3 levels using an MSD ELISA assay. See FIG. 6C.

There was a marked reduction in the levels of H3K27me3 in Inhibitor 1 treated tumors compared to the vehicle control, and similar reduction was observed in the tumors from mice treated with Inhibitor 1 and lenalidomide combination as compared to lenalidomide alone. There was no additional reduction in H3K27me3 levels observed in the Inhibitor 1 and lenalidomide combination tumors as compared to tumors treated with Inhibitor 1 alone. Lenalidomide treated tumors did not affect the H3K27me3 levels. Total H3 was used as a normalizer. Data are represented as the mean of data from 4 subjects±s.e.m.

### Synergism Between EZH2 Inhibitors and PI3K Inhibitors (Idelalisib and Buparlisib)

KARPAS-422 cells were treated with different doses of Inhibitor 1 for 4 days followed by another 3 days of cotreatment with Inhibitor 1 and idelalisib (CAL-101) or buparlisib (B KM-120).

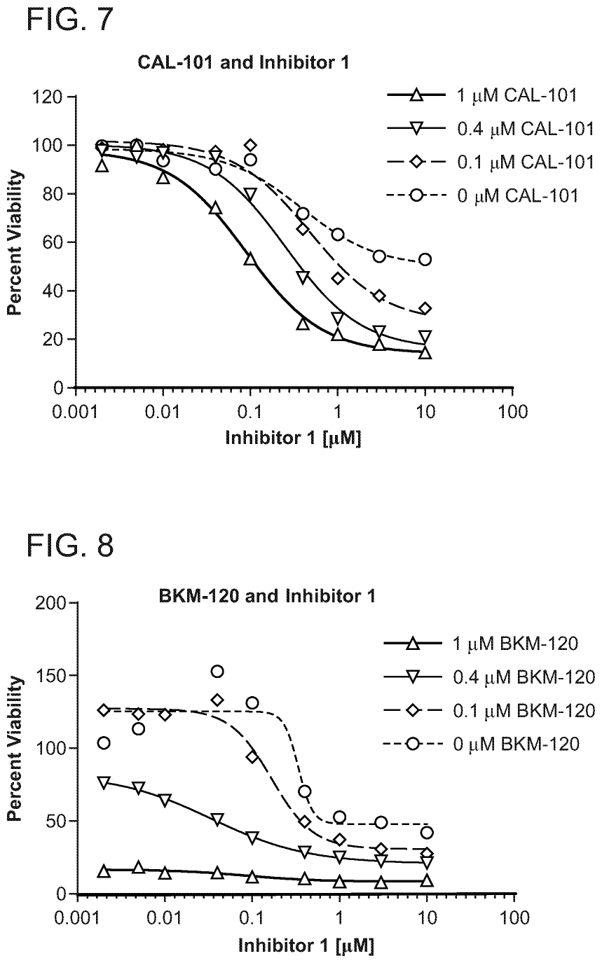

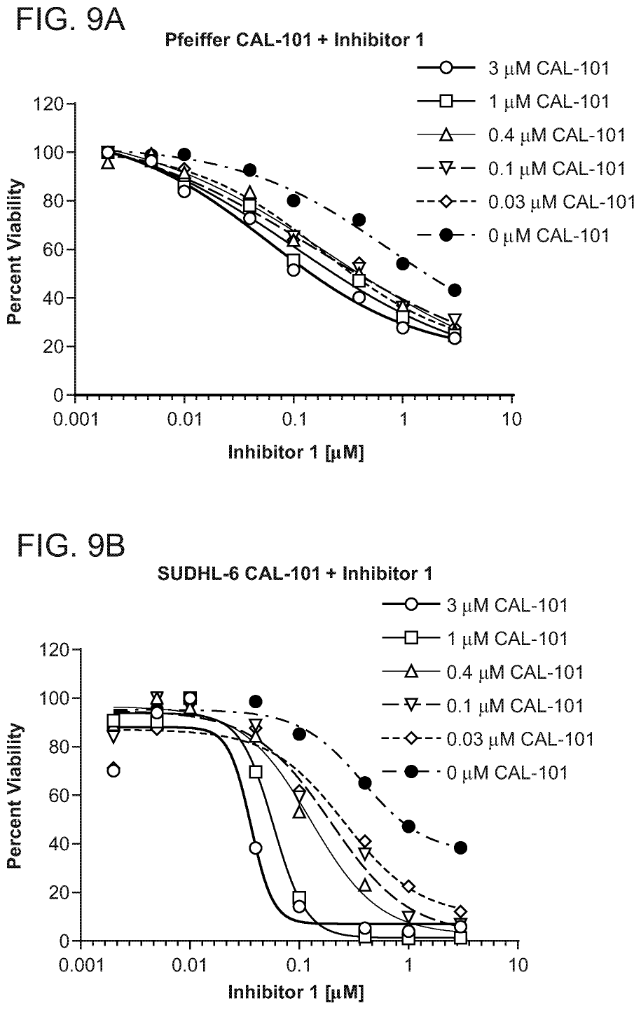

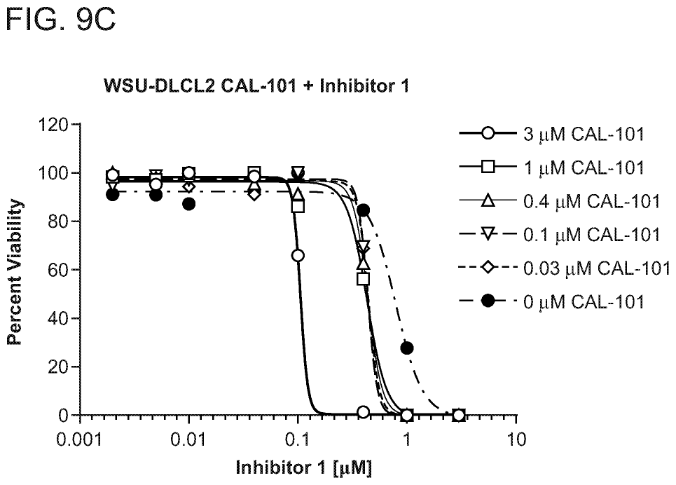

Viability was measured using Cell Titre Glo. See FIGS. 7-9. A significant decrease in cell viability, i.e., growth inhibition, was seen in lymphoma cell lines (KARPAS-422, Pfeiffer, SU-DHL6 and WSU-DLCL2) with both Inhibitor 1 and idelalisib or buparlisib.

### Synergism Between EZH2 Inhibitors and Histone Deacetylase Inhibitors (Panobinostat)

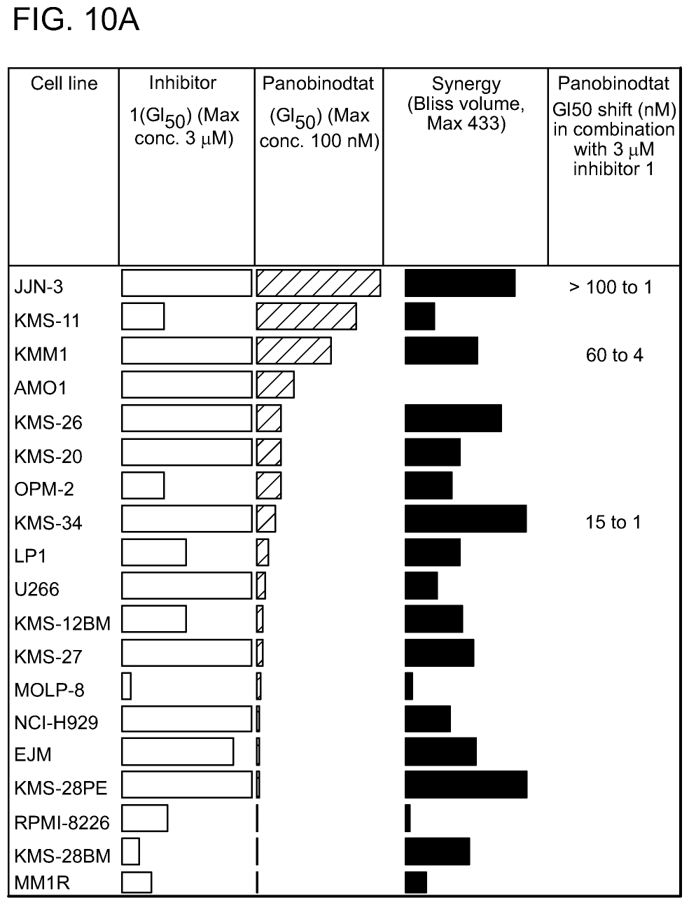

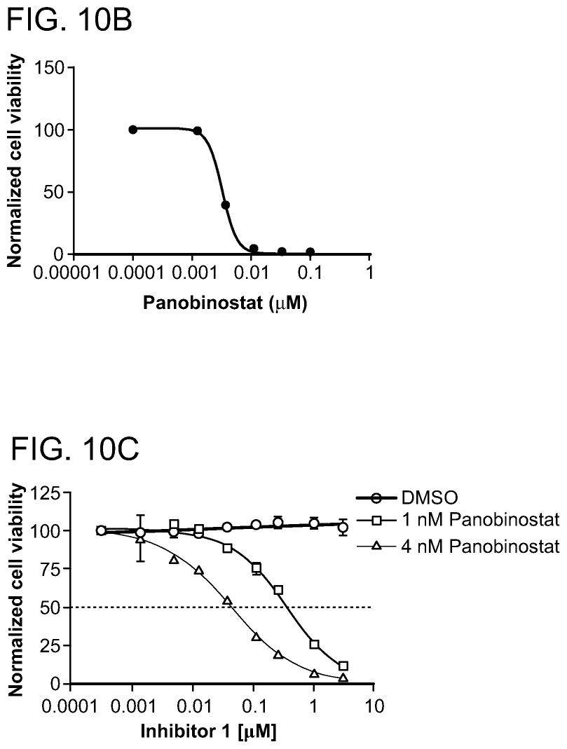

19 different multiple myeloma cell lines were tested for EZH2 inhibitor and HDAC inhibitor (Panobinostat) combination as shown in FIG. 10A. Multiple myeloma cells were pre-treated with Inhibitor 1 for 8 days. This was followed by co-treatment of Inhibitor 1 and Panobinostat for an additional 2 days and viability was assessed at the end of the experiment. FIG. 10B-C shows the dose response data for KMS-28PE cell line where the two inhibitors synergise with each other in causing cell death. The results showed a strong synergistic effect between the two inhibitors. Next, the combination of Inhibitor 1 and Panobinostat were treated in an OPM-2 xenograft model.

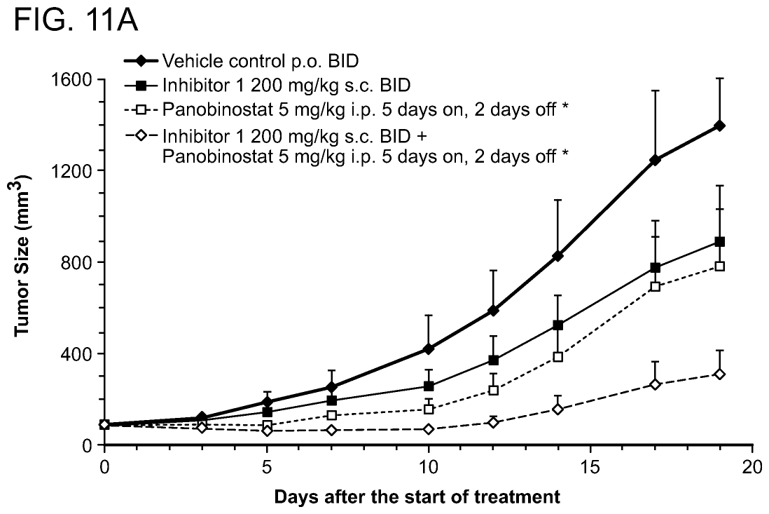

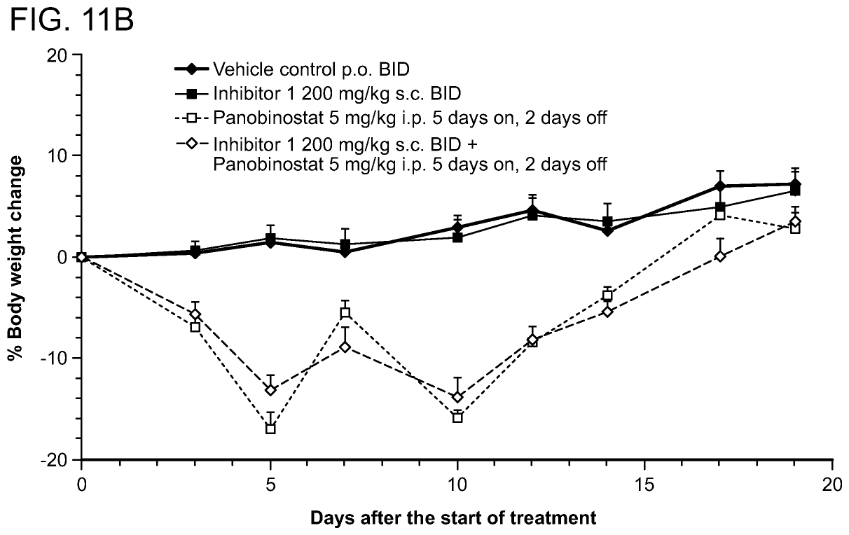

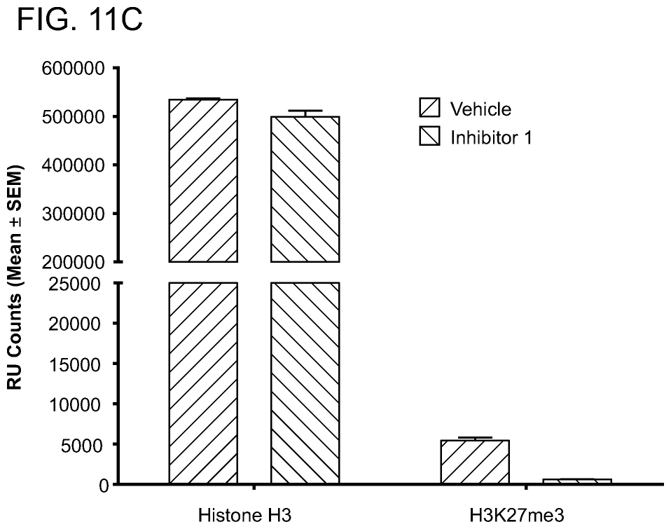

FIG. 11A is an in vivo combination experiment using an OPM-2 subcutaneous xenograft BALB/C nude mouse model (n=8 per cohort). The experiment was carried out to assess the impact of Inhibitor 1 and Panobinostat in combination. Inhibitor 1 was dosed twice daily at 200 mg/kg administered subcutaneously (200 mpk SC BID) and Panobinostat was dosed intraperitoneally once a day. Panobinostat was dosed at 5 mg/kg QD (DO-D4); 2.5 mg/kg QD (D7-D9, D13, D17 and D19), the dose and frequency of dosing was reduced to manage the toxicity effects. Tumor volume was plotted as a function of time. Data are represented as the mean tumor volume per cohort and time point±s.d. Bodyweight was assessed for vehicle and compound-treated animals for the experiment described in FIG. 11B. Data are represented as described in FIG. 11C. Vehicle and Inhibitor 1-treated tumors were collected at the end of the study and analyzed for global H3K27me3 and total H3 levels using an MSD ELISA assay. See FIG. 11D. There was a marked reduction in the levels of H3K27me3 in Inhibitor 1 treated tumors compared to the vehicle control. Data are represented as the mean of data from 4 subjects±s.e.m.

### Synergism Between EZH2 Inhibitors and LSD1 Inhibitors

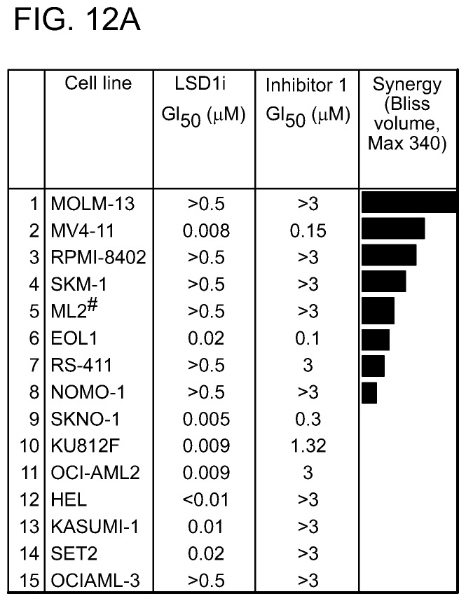

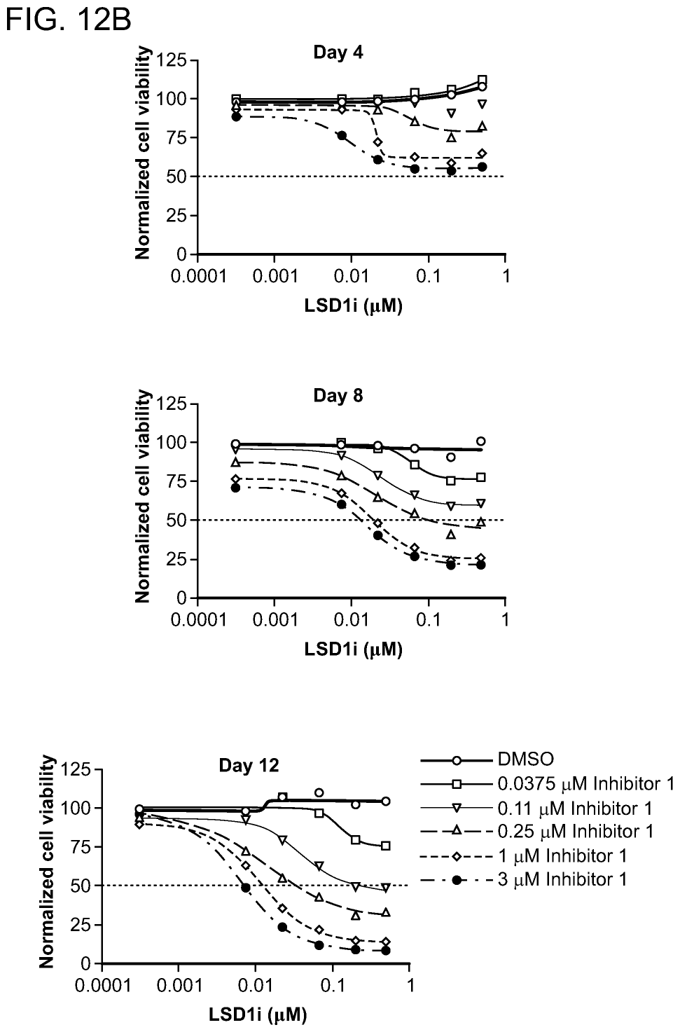

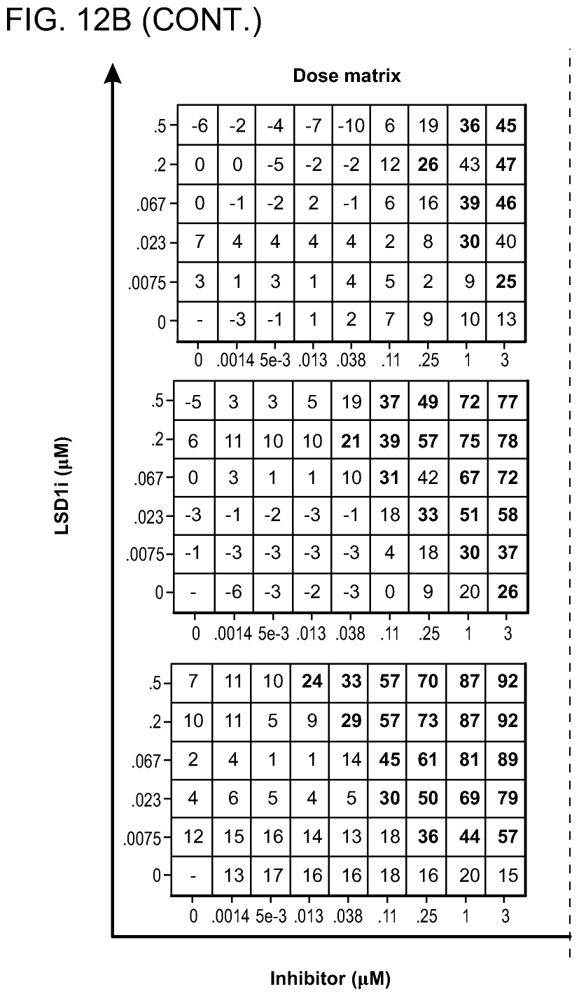

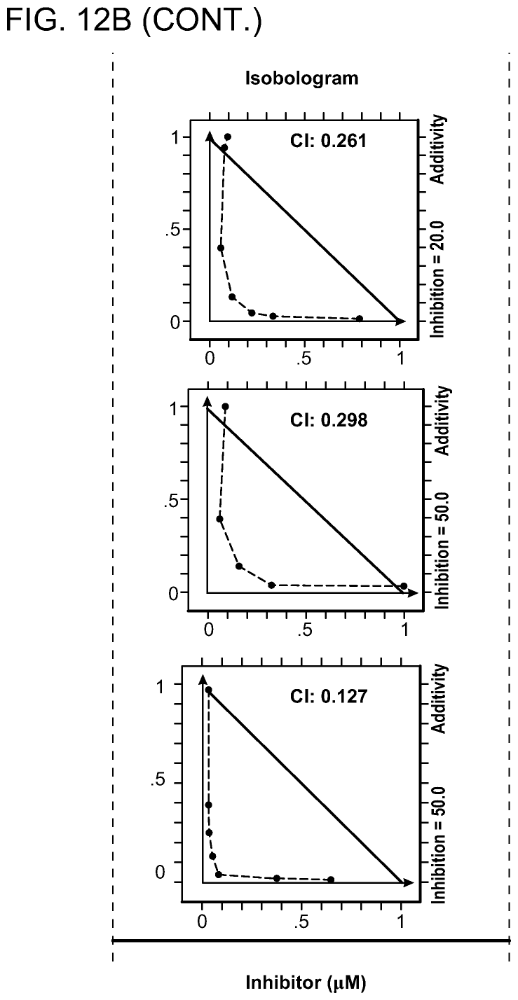

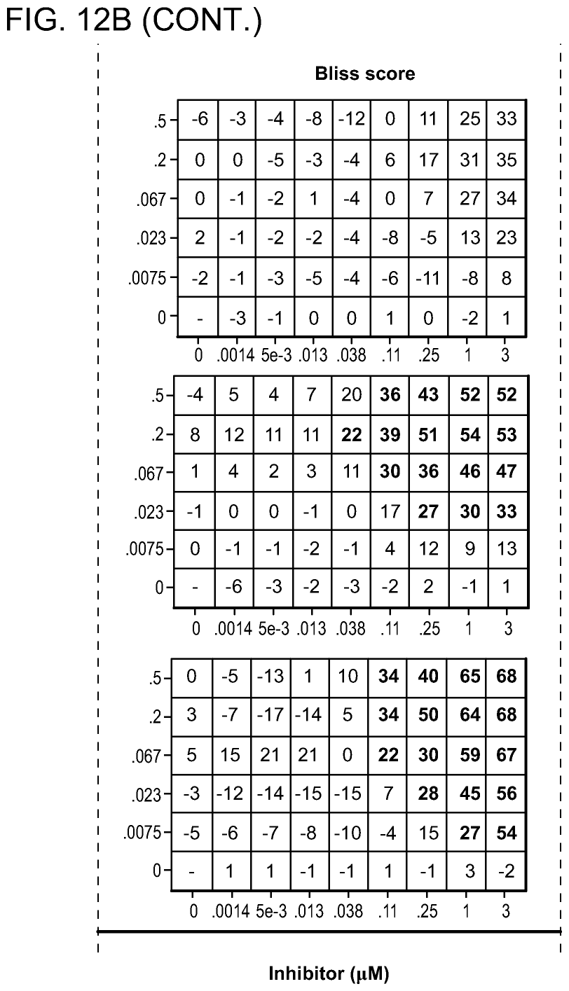

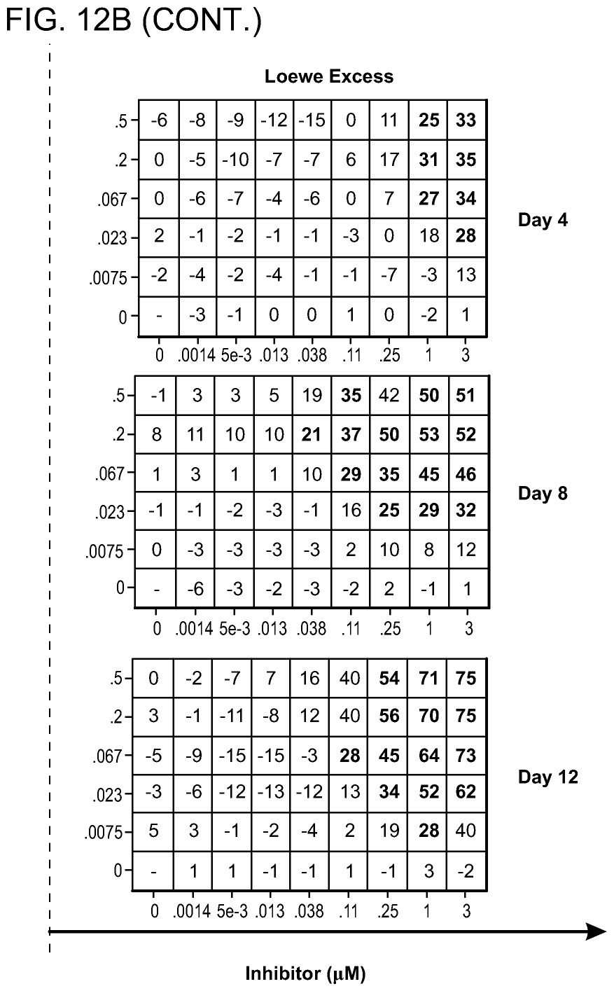

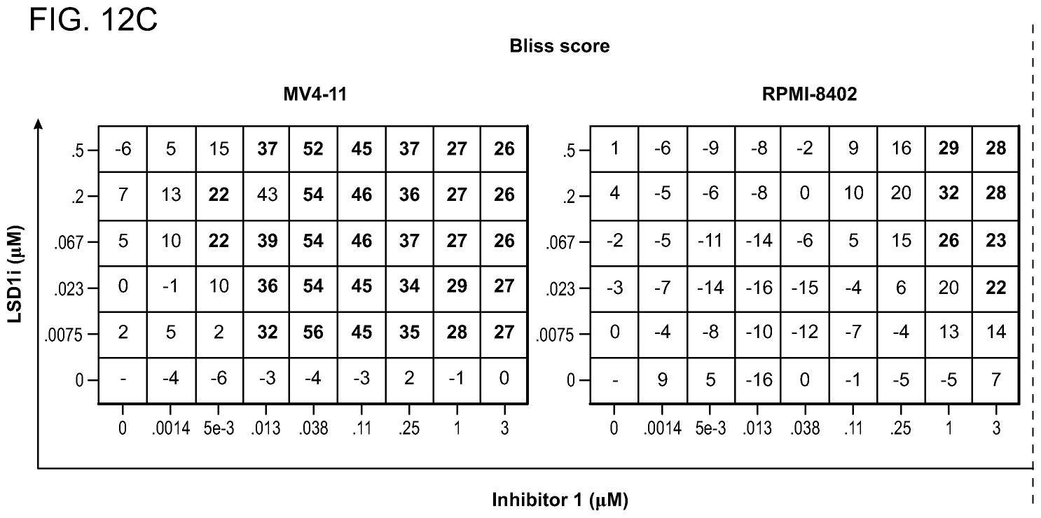

15 different leukemia cell lines were tested for EZH2 inhibitor and LSD1 inhibitor (RN-1) combination as shown in FIG. 12A. Cells were pre-treated with Inhibitor 1 for 4 days. This was followed by co-treatment of Inhibitor 1 and LSD1 inhibitor for an additional 12 days and viability was assessed after every 4 days of co-treatmentt. FIG. 12B shows the dose response data for MOLM-13 cell line where the two inhibitors synergise with each other in causing cell death. The results showed a strong synergistic effect between the two inhibitors. FIG. 12B (bottom) shows the synergy score calculated using various different matrices. FIG. 12C shows the bliss scores for several other leukemia cell lines showing synergy between Inhibitor 1 and LSD1 inhibitors.

### Synergism Between EZH2 Inhibitors and Anti-Inflammatory Steroids (Dexamethasone)

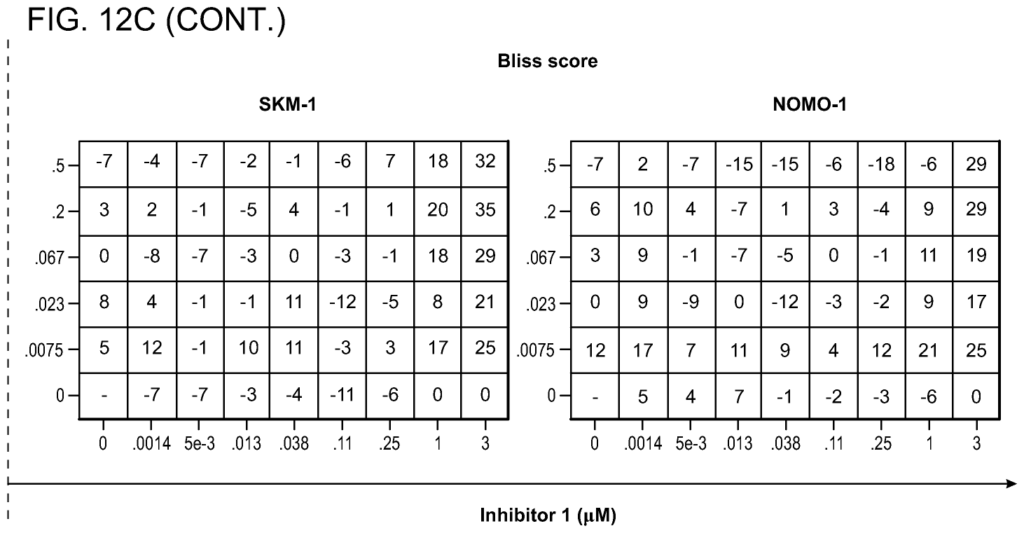

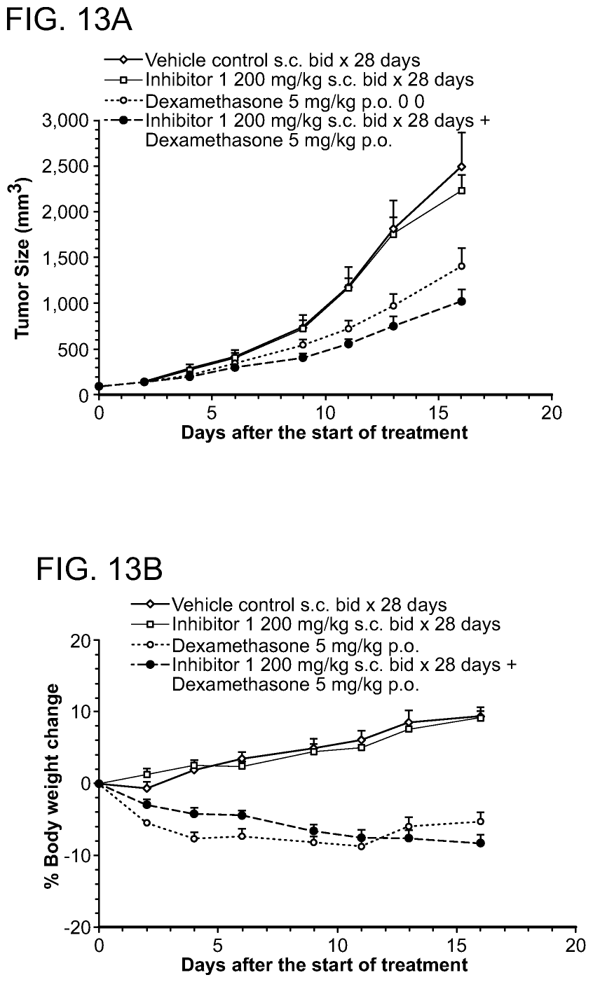

EZH2 Inhibitor 1 and dexamethasone act synergistically and reduce tumor growth in the OPM-2 xenograft model. Inhibitor 1 has little single agent activity in the OPM-2 xenograft model. However, Inhibitor 1 increases the in vivo anti-tumor activity of dexamethasone. See FIG. 13 where FIG. 13A is an in vivo combination experiment using an OPM-2 subcutaneous xenograft BALB/C nude mouse model (n=8 per cohort; this experiment was run as a part of the experiment described in FIG. 6). The experiment was carried out to assess the impact of Inhibitor 1 and dexamethasone in combination. Inhibitor 1 was dosed twice daily at 200 mg/kg administered subcutaneously (200 mpk SC BID) and dexamethasone was dosed orally at 5 mg/kg (5 days on 2 days off) for the entire course of the study. Tumor volume was plotted as a function of time. Data are represented as the mean tumor volume per cohort and time poin±s.d. Bodyweight was assessed for vehicle and compound-treated animals for the experiment described for FIG. 6. See FIG. 13B. No significant adverse effects were observed during the course of the experiment. Data are represented as described in FIG. 13B.

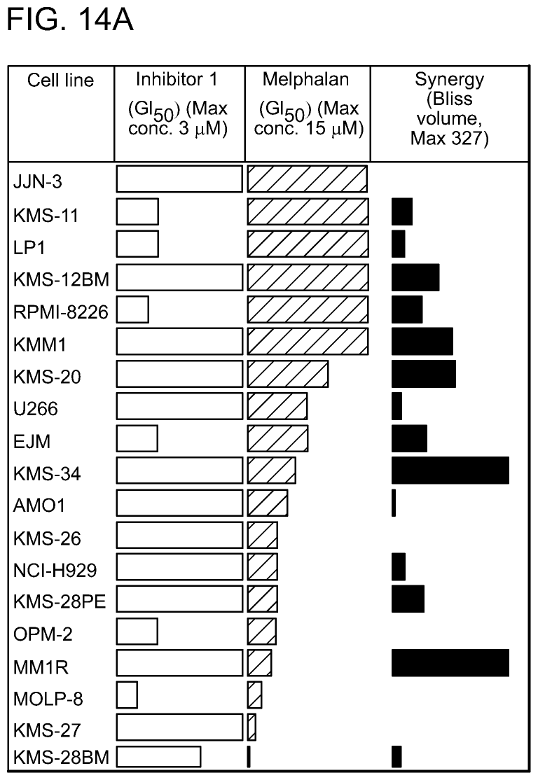

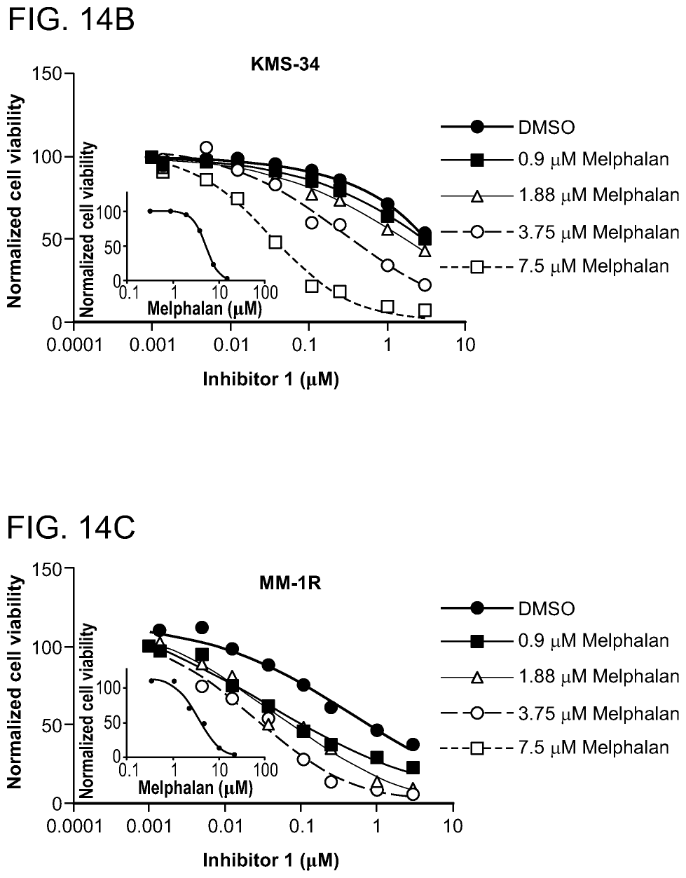

19 different multiple myeloma cell lines were tested for EZH2 inhibitor and Melphalan combination as shown in FIG. 14A. Multiple myeloma cells were pre-treated with Inhibitor 1 for 8 days. This was followed by co-treatment of Inhibitor 1 and Panobinostat for an additional 4 days and viability was assessed at the end of the experiment. FIG. 14B-C shows the dose response data for KMS-34 and MM-1R cell lines respectively where the two inhibitors synergise with each other in causing cell death. The results showed a strong synergistic effect between the two inhibitors in both the cell lines.

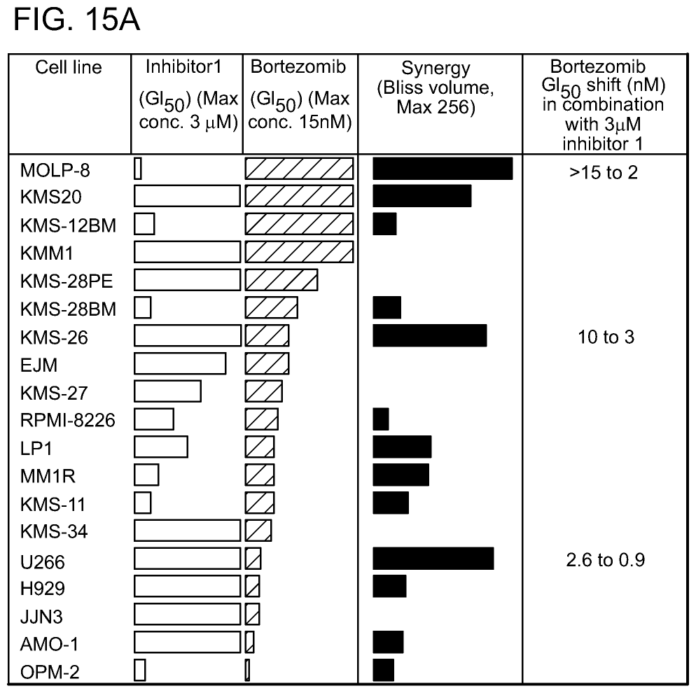

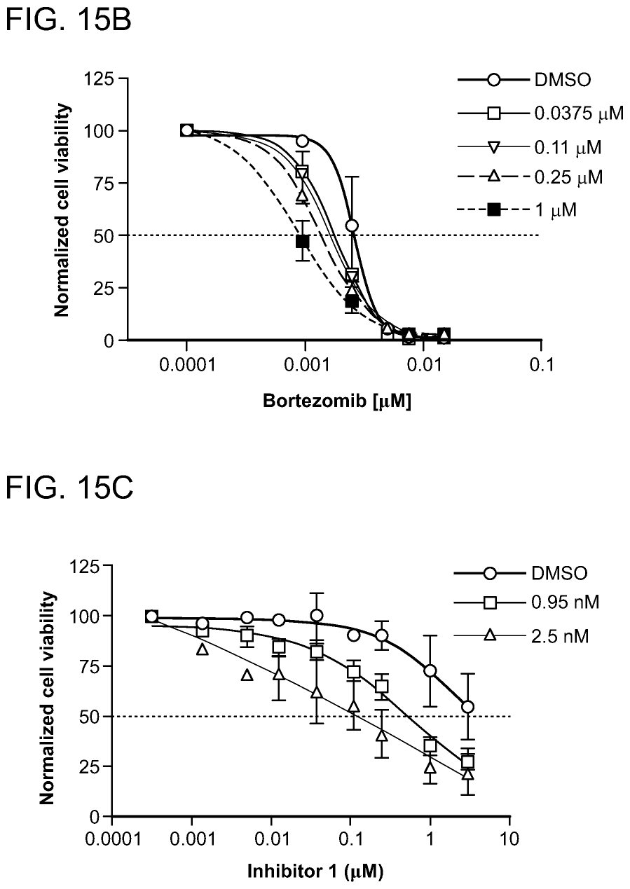

Similarly, we also tested the combination of Inhibitor 1 and Bortezomib in a panel of multiple myeloma cell lines. Cells were pre-treated with Inhibitor 1 for 8 days. This was followed by co-treatment of Inhibitor 1 and Bortezomib for an additional 2 days and viability was assessed at the end of the experiment. FIG. 15A summarizes the data for the combination. FIG. 15B-C show the dose response curves for U266 cell line to show a synergistic effect between the two inhibitors.

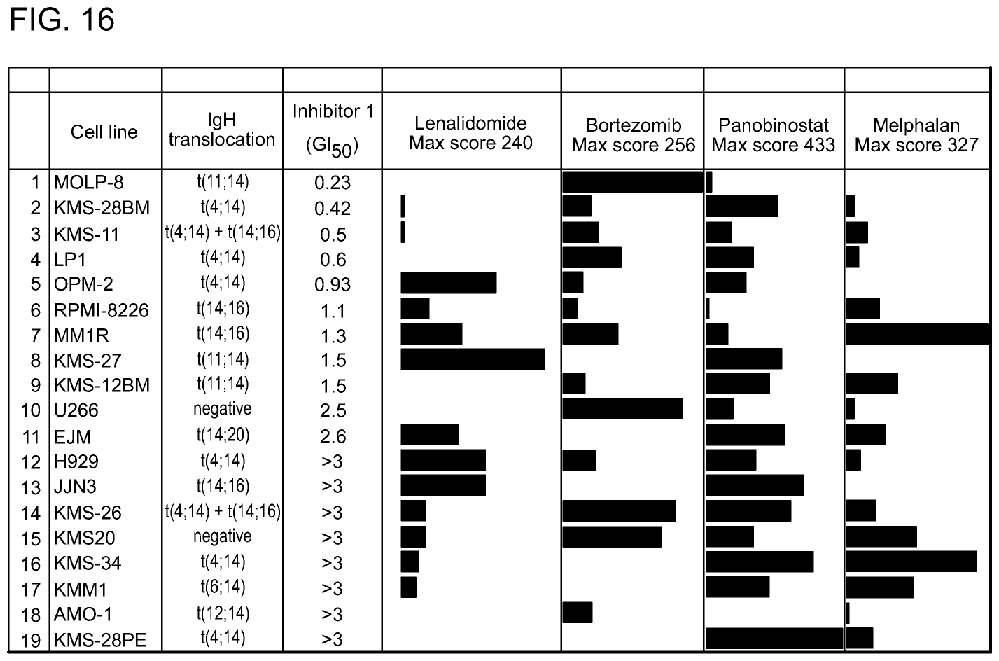

FIG. 16 summarizes the synergies observed between various standard of care agents as described above in 19 multiple myeloma cell lines. The data shows that Inhibitor 1 broadly combines with various different standard of care agents and combining EZH2 inhibitors with these standard of care agents could provide clinical efficacy.

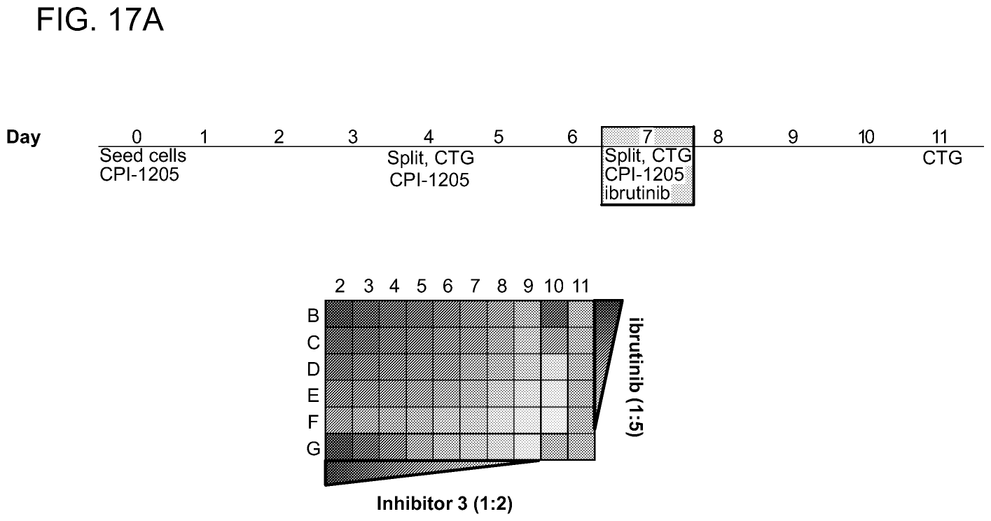

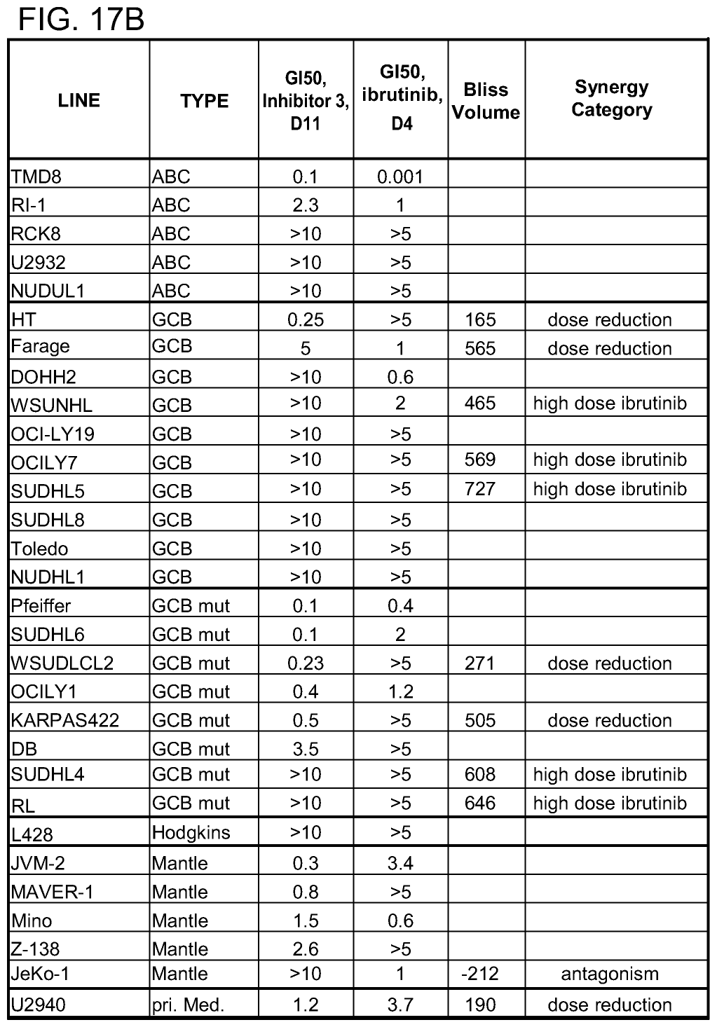

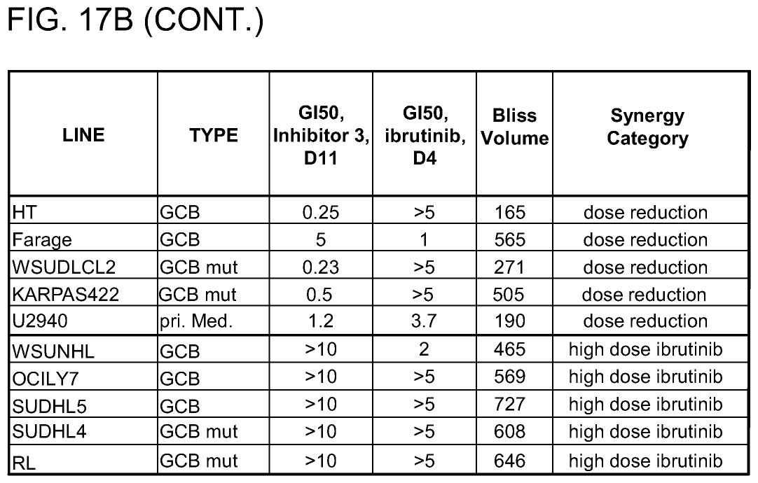

FIG. 17 summarizes the results from combination of Inhibitor 3 and Ibrutinib. Experiment was performed as outlined in FIG. 17A. The results and the bliss volumes for each of the lymphoma cell lines are summarized in FIG. 17B.

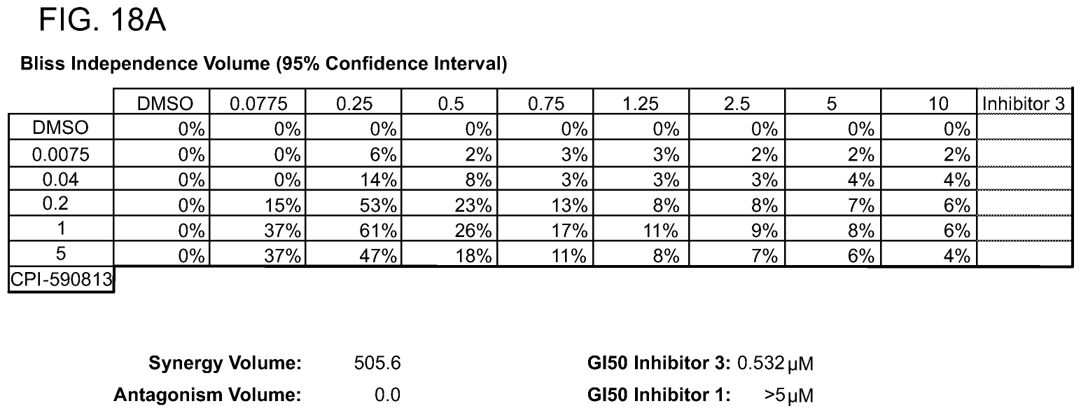

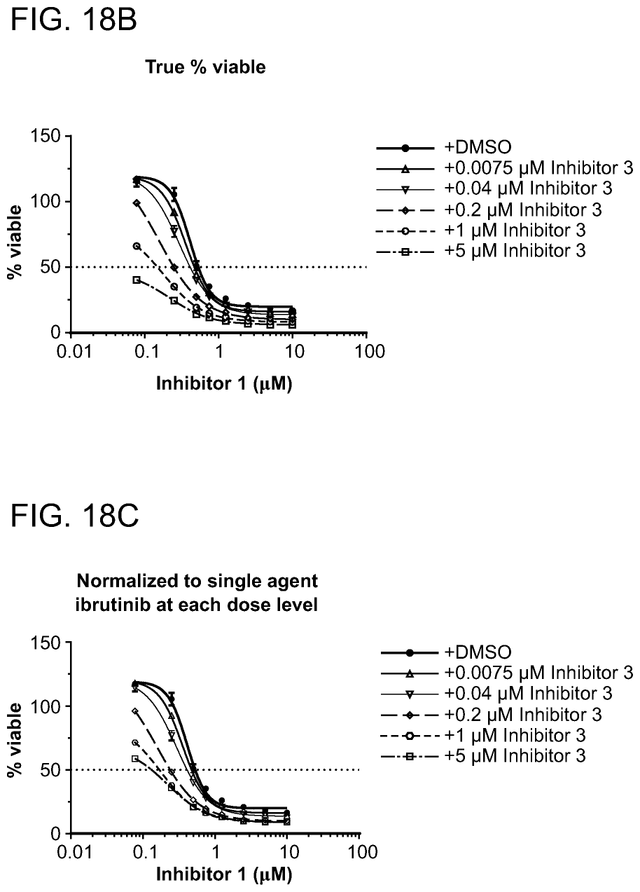

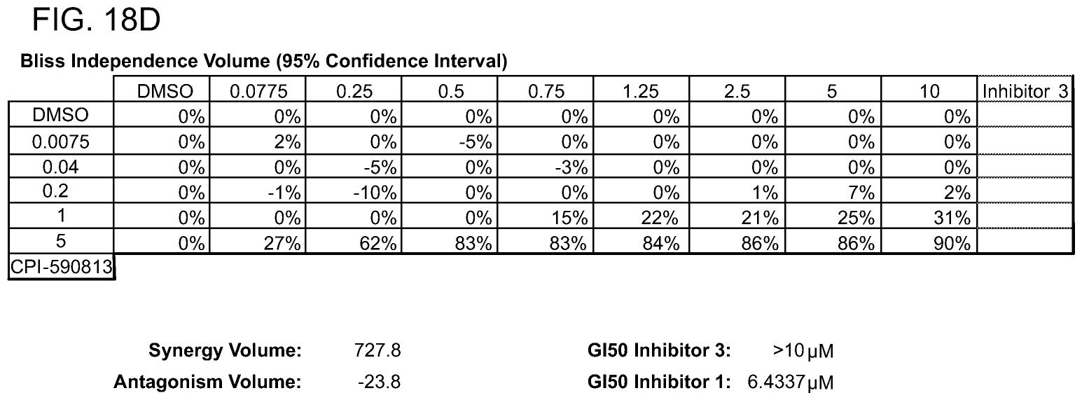

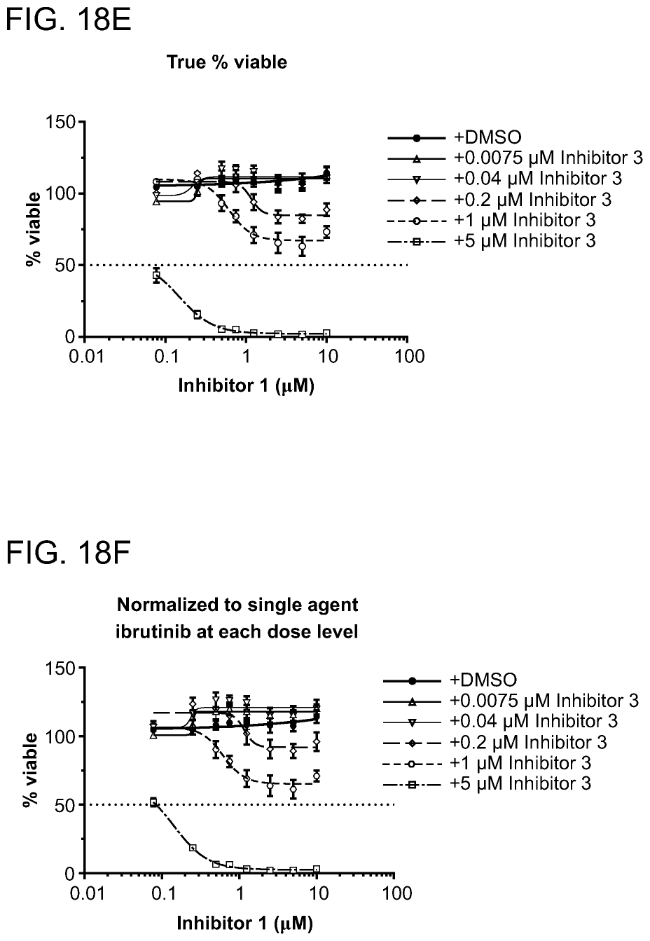

FIG. 18 illustrates the synergy observed between Inhibitor 3 and Ibrutinib in KARPAS-422 and SUDHL5 cell lines, FIGS. 18A-C and FIGS. 18D-F, respectively.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating cancer in a subject in need thereof comprising the step of administering to the subject in need thereof an effective amount of an EZH2 inhibitor and an effective amount of second agent selected from a phthalimide; an anti-inflammatory steroid; a PI3K inhibitor; an LSD1 inhibitor; a histone deacetylase inhibitor; cyclophosphamide, hydroxydaunorubicin, oncovin, and prednisone (CHOP); rituximab and CHOP; prednisolone;
bortezomib; a bromodomain-targeted compound; and ibrutinib.

2. A method of treating cancer in a subject in need thereof comprising the step of administering to the subject in need thereof an effective amount of an EZH2 inhibitor and an effective amount of an alkylating antineoplastic agent.

3. The method of claim 1, wherein the second agent is selected from a phthalimide, an anti-inflammatory steroid, a PI3K inhibitor, an LSD1 inhibitor, a histone deacetylase inhibitor, and CHOP.

4. The method of claim 1, wherein the second agent is the phthalimide lenalidomide.

5. The method of claim 1, wherein the second agent is the anti-inflammatory steroid dexamethasone.

6. The method of claim 1, wherein the second agent is the PI3K inhibitor idelalisib or buparlisib.

7. The method of claim 1, wherein the second agent is the histone deacetylase inhibitor panobinostat.

8. The method of claim 1, wherein the second agent is the LSD1 inhibitor [RN-1].

9. The method of claim 1, wherein the the second agent is CHOP.

10. The method of claim 2, wherein the alkylating antineoplastic agent is melphalan.

11. The method of claim 1, wherein the EZH2 inhibitor is selected from 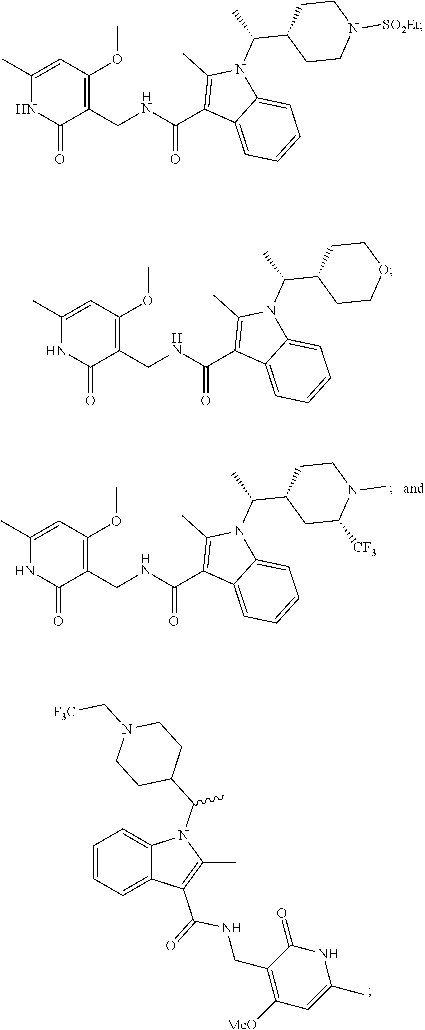
or a pharmaceutically acceptable salt thereof.

12. The method of claim 1, wherein the EZH2 inhibitor is 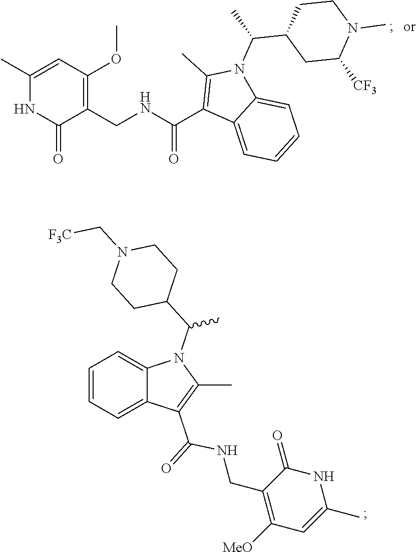
or a pharmaceutically acceptable salt thereof.

13. The method of claim 1, wherein the EZH2 inhibitor is selected from EPZ005687, EPZ011989, EI1, GSK126, GSK343, UNC1999, and EPZ-6438.

14. The method of claim 1, wherein the EZH2 inhibitor is administered concurrently with the second agent.

15. The method of claim 1, wherein the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL).

16. The method of claim 1, wherein the cancer is selected from melanoma, prostate cancer, breast cancer, ovarian cancer, colon cancer, bladder cancer, lung adenocarcinoma, and carcinoma of the pancreas.

17. A pharmaceutical composition comprising an effective amount of an EZH2 inhibitor together with a phthalimide; an anti-inflammatory steroid; an anti-OX40 antibody; a PI3K inhibitor; an LSD1 inhibitor; a histone deacetylase inhibitor; cyclophosphamide, hydroxydaunorubicin, oncovin, and prednisone (CHOP); rituximab and CHOP; prednisolone; bortezomib; a bromodomain-targeted compound; or ibrutinib.

18. A pharmaceutical composition comprising an effective amount of an EZH2 inhibitor together with an alkylating antineoplastic agent.

19. The pharmaceutical composition of claim 17, wherein the composition comprises an effective amount of an EZH2 inhibitor together with a phthalimide, an anti-inflammatory steroid, a PI3K inhibitor, an LSD1 inhibitor, a histone deacetylase inhibitor, or CHOP.

20. The pharmaceutical composition of claim 17, wherein the composition comprises an EZH2 inhibitor together with the phthalimide lenalidomide.

21. The pharmaceutical composition of claim 17, wherein the composition comprises an EZH2 inhibitor together with anti-inflammatory steroid dexamethasone.

22. The pharmaceutical composition of claim 17, wherein the composition comprises an EZH2 inhibitor together with the PI3K inhibitor idelalisib or buparlisib.

23. The pharmaceutical composition of claim 17, wherein the composition comprises an EZH2 inhibitor together with the histone deacetylase inhibitor panobino stat.

24. The pharmaceutical composition of claim 17, wherein the composition comprises an EZH2 inhibitor together with the LSD1 inhibitor [RN-1], ORY-1001, or GSK2879552.

25. The pharmaceutical composition of claim 17, wherein the composition comprises an EZH2 inhibitor together with the LSD1 inhibitor [RN-1].

26. The pharmaceutical composition of claim 17, wherein the composition comprises an EZH2 inhibitor together with CHOP.

27. The pharmaceutical composition of claim 18, wherein the composition comprises an EZH2 inhibitor together with melphalan.

28. The pharmaceutical composition of claim 17, wherein the EZH2 inhibitor is selected from 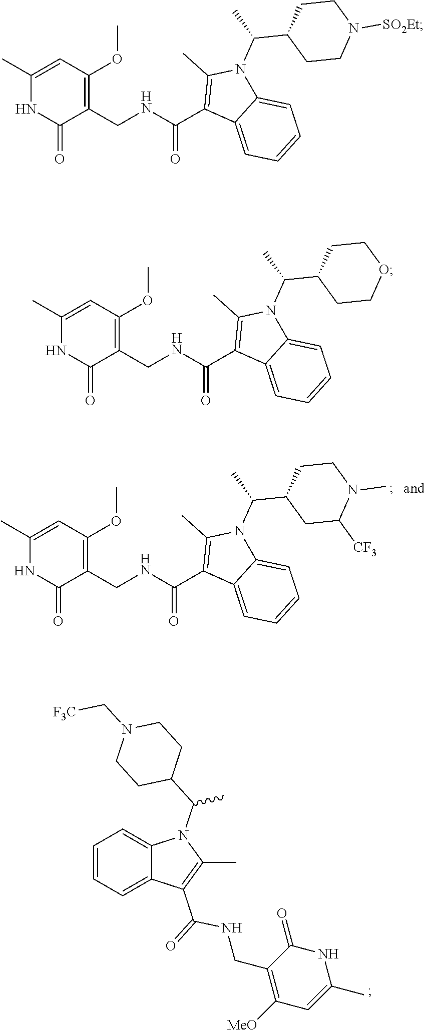
or a pharmaceutically acceptable salt thereof.

29. The pharmaceutical composition of claim 17, wherein the EZH2 inhibitor is 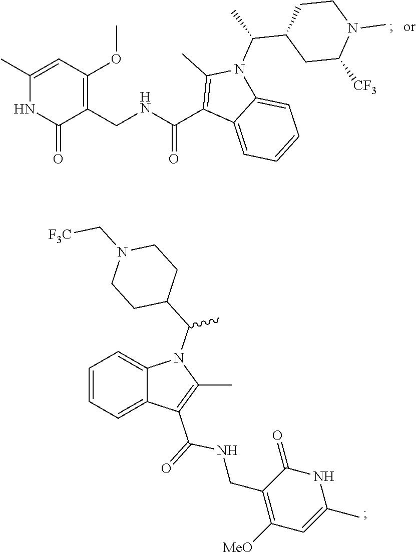
or a pharmaceutically acceptable salt thereof.

30. The pharmaceutical composition of claim 17, wherein the EZH2 inhibitor is selected from EPZ005687, EPZ011989, EI1, GSK126, GSK343, UNC1999, and EPZ-6438.
